(19) United States
(12) Patent Application Publication
(10) Pub. No.: US 2023/0089738 A1
(43) Pub. Date: Mar. 23, 2023

(54) DEVICES AND METHODS FOR SECURING PROSTHETIC IMPLANTS WITHIN A PATIENT'S VASCULATURE

(71) Applicant: Edwards Lifesciences Corporation, Irvine, CA (US)

(72) Inventors: Adi Carmi, Ganei Tikva (IL); Boaz Manash, Givat Ada (IL); Nikolai Gurovich, Hadera (IL); Michael Bukin, Pardes Hanna (IL)

(21) Appl. No.: 17/992,730

(22) Filed: Nov. 22, 2022

Related U.S. Application Data
(63) Continuation of application No. PCT/US2021/034399, filed on May 27, 2021.
(60) Provisional application No. 63/030,811, filed on May 27, 2020.

(51) Int. Cl. A61F 2/24

(52) U.S. Cl. CPC A61F 2/2418; A61F 2220/0016; A61F 2002/0081

(57) ABSTRACT

A prosthetic valve assembly includes a prosthetic valve and an anchoring frame. The prosthetic valve includes a valve frame and a valve structure. The valve frame includes a plurality of struts and is expandable from a compressed configuration to an expanded configuration. The valvular structure includes leaflets. The valvular structure is disposed radially within and is coupled to the valve frame. The valvular structure is configured to allow blood flow through the prosthetic valve assembly in a first direction and to restrict blood flow through the prosthetic valve assembly in a second direction. The anchoring frame includes a plurality of struts. The anchoring frame is disposed radially outwardly from and is coupled to the valve frame. The struts of the anchoring frame comprise one or more tissue-engaging elements configured for contacting native tissue to help secure the prosthetic valve assembly at an implantation location.

### CROSS-REFERENCE TO RELATED APPLICATIONS

This application is a continuation of International Application No. PCT/US2021/034399, filed May 27, 2021, which claims the benefit of U.S. Provisional Application No. 63/030,811, filed May 27, 2020. The prior applications are incorporated by reference herein in their entirety.

### FIELD

The present disclosure generally concerns implantable prosthetic devices and more particularly devices and related methods for securing prosthetic devices relative to native tissue within a patient's vasculature.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable.

In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Once expanded, the prosthetic heart valve contacts the surrounding native heart valve tissue to secure the prosthetic heart valve in place. The condition of the native heart valve tissue can vary widely from patient to patient. Also, the anatomy of the various native valves of a heart varies greatly. In additional to prosthetic heart valve, there are other types of prostheses for treating issues with a patient's vasculature system. These devices can include stents, valve docking frames, grafts, to name a few. As such, there is continuing need for prosthetic heart valves and other prostheses that are suitable for the variation in patients and/or that can be used for various implantation locations.

### SUMMARY

Described herein are prosthetic valves and other prostheses and methods for implanting the prosthetic valves and prostheses. In some examples, a prosthetic valve assembly is disclosed. The disclosed prosthetic valve assemblies have anchoring frames and/or other features coupled to the valve frame that are configured for securing the prosthetic valves to the native tissue. In some example, the anchoring frames can comprise tissue-engaging elements configured to increase friction between the prosthetic valve assembly and the native tissue. As such, the prosthetic valve assemblies disclosed herein can resist migration relative to the native tissue. The disclosed assemblies can be used, for example, at implantation locations that would not provide sufficient structure for a typical prosthetic valve to be secured therein. For example, these assemblies can be implanted at a native heart valve, including an aortic valve, a mitral valve, a tricuspid valve, and/or a pulmonary valve). In particular, the disclosed prosthetic valves can be implanted in non-stenotic and/or non-calcified native valves. Additionally or alternatively, the prosthetic valves disclosed herein can be implanted at other locations within a patient's vasculature, such as a blood vessel (e.g., a vena cava).

In one representative example, a prosthetic heart valve assembly includes a prosthetic heart valve and an anchoring frame. The prosthetic heart valve includes a valve frame and a valve structure. The valve frame includes a plurality of struts and is expandable from a radially compressed delivery configuration to a radially expanded configuration. The valvular structure includes a plurality of leaflets. The valvular structure is disposed radially within and is coupled to the valve frame. The valvular structure is configured to allow blood flow through the prosthetic heart valve assembly in a first direction and to restrict blood flow through the prosthetic heart valve assembly in a second direction. The anchoring frame includes a plurality of struts. The anchoring frame is disposed radially outwardly from and is coupled to the valve frame. The struts of the anchoring frame comprise one or more tissue-engaging elements configured for contacting native tissue to help secure the prosthetic heart valve assembly at an implantation location.

In another representative example, an anchoring frame for a prosthetic heart valve assembly is provided. The anchoring frame includes a plurality of interconnected struts and a plurality of projections extending from the struts. The struts are configured to be moved from a radially compressed configuration to a radially expanded configuration, and the struts are configured to be coupled to a prosthetic heart valve. The projections are configured to engage native tissue at an implantation location.

In another representative example, an anchoring frame includes a plurality of interconnected struts comprising a plurality of grooves formed in radially outwardly facing surfaces of the struts. The struts are configured to be coupled to a prosthetic heart valve, the anchoring frame can be expanded from a radially compressed configuration to a radially expanded configuration, and the grooves are configured to receive native tissue therein when the anchoring frame is radially expanded at an implantation location.

In another representative example, an anchoring frame comprises a plurality of interconnected struts and a plurality of tines extending from the struts. The struts are configured to be moved from a radially compressed configuration to a radially expanded configuration, and the struts are configured to be coupled to a prosthetic heart valve. The tines are arranged in pairs including a first tine and a second tine. The first and second tine are axially aligned with each other. The first tine and the second tine are axially spaced apart relative to each other by a first distance when the struts are in the radially compressed configuration. The first tine and the second tine are axially spaced apart relative to each other by a second distance when the struts are in the radially expanded configuration, and the first distance is greater than the second distance.

In another representative example, the anchoring frame comprises a plurality of interconnected struts and a plurality of tines. The struts are configured to be moved from a radially compressed configuration to a radially expanded configuration, and the struts are configured to be coupled to a prosthetic heart valve. Each tine comprises a fixed end portion and a free end portion. The fixed end portions are coupled to one or more of the struts, and the free end portions are movable relative to the struts. When the struts are in the radially compressed configuration, the free end portions of the tines are in a first radial position. When the struts are in the radially expanded configuration, the free end portions of the tines are in a second radial position. The second radial position is farther from a central longitudinal axis of the anchoring frame than the first radial position.

In another representative example, a prosthetic heart valve includes a valve frame, a valvular structure, and one or more friction-increasing elements. The valve frame comprises a plurality of struts and is expandable from a radially compressed delivery configuration to a radially expanded configuration. The valvular structure comprises a plurality of leaflets and is disposed radially within and is coupled to the valve frame. The valvular structure is configured to allow blood flow through the prosthetic heart valve assembly in a first direction and to restrict blood flow through the prosthetic heart valve assembly in a second direction. The friction-increasing elements are wrapped around the struts of the frame and are configured to engage native tissue and help to prevent the prosthetic heart valve from migrating relative to the native tissue.

In another representative example, a prosthetic heart valve includes a valve frame, a valvular structure, and one or more friction-increasing elements. The valve frame comprises a plurality of struts and is expandable from a radially compressed delivery configuration to a radially expanded configuration. The valvular structure comprises a plurality of leaflets and is disposed radially within and is coupled to the valve frame. The valvular structure is configured to allow blood flow through the prosthetic heart valve assembly in a first direction and to restrict blood flow through the prosthetic heart valve assembly in a second direction. The friction-increasing elements circumscribe the valve frame, and the friction-increasing elements are configured to engage native tissue and help to prevent the prosthetic heart valve from migrating relative to the native tissue.

In another representative example, a prosthetic heart valve includes a valve frame and a valve structure. The valve frame comprises a plurality of struts and a plurality of anchoring members and is expandable from a radially compressed delivery configuration to a radially expanded configuration. The valve frame comprises a cylindrical shape in the radially compressed configuration and a non-cylindrical shape in the radially expanded configuration. The struts elastically deform when the valve frame moves from the radially compressed configuration to the radially expanded configuration, and the anchoring members plastically deform when the valve frame moves from the radially compressed configuration to the radially expanded configuration. The valvular structure includes a plurality of leaflets. The valvular structure is disposed radially within and is coupled to the valve frame. The valvular structure is configured to allow blood flow through the prosthetic heart valve assembly in a first direction and to restrict blood flow through the prosthetic heart valve assembly in a second direction.

In another representative example, a prosthetic heart valve includes an inner frame, a valve structure, and an outer frame. The inner frame includes a plurality of struts, and the inner frame is expandable from a radially compressed delivery configuration to a radially expanded configuration. The valvular structure includes a plurality of leaflets. The valvular structure is disposed radially within and is coupled to the inner frame. The valvular structure is configured to allow blood flow through the prosthetic heart valve in a first direction and to restrict blood flow through the prosthetic heart valve in a second direction. The outer frame includes a plurality of struts. The outer frame is disposed radially outwardly from and is coupled to the inner frame. The struts of the outer frame comprise one or more tissue-engaging elements configured for contacting native tissue to help secure the prosthetic heart valve at an implantation location.

In another representative example, a method for implanting a prosthetic heart valve or a prosthetic heart valve assembly is provided. The method includes releasably coupling the prosthetic heart valve or the prosthetic heart valve assembly to a delivery apparatus, inserting the delivery apparatus and the prosthetic heart valve or the prosthetic heart valve assembly into a patient's vasculature, advancing the prosthetic heart valve or the prosthetic heart valve assembly to an implantation location, expanding the prosthetic heart valve or the prosthetic heart valve assembly from the radially compressed configuration to the radially expanded configuration, wherein the prosthetic heart valve or the prosthetic heart valve assembly contacts native tissue at the implantation location, and releasing the prosthetic heart valve or the prosthetic heart valve assembly from the delivery apparatus.

In another representative example, a prosthetic valve includes an inner frame, a valve structure, and an outer frame. The inner frame includes a plurality of struts, and the inner frame is expandable from a radially compressed delivery configuration to a radially expanded configuration. The inner frame comprises a first thickness. The valvular structure includes a plurality of leaflets. The valvular structure is disposed radially within and is coupled to the inner frame. The valvular structure is configured to allow blood flow through the prosthetic valve in a first direction and to restrict blood flow through the prosthetic valve in a second direction. The outer frame includes a plurality of struts. The outer frame is disposed radially outwardly from and is coupled to the inner frame. The struts of the outer frame comprise one or more tissue-engaging elements configured for contacting native tissue to help secure the prosthetic valve at an implantation location. The outer frame comprises a second thickness which is less than the first thickness of the inner frame.

In another representative example, a docking assembly comprises a prosthetic docking device and an anchoring frame. The prosthetic docking device including a frame configured for receiving and supporting a prosthesis therein. The anchoring frame coupled to the frame of the prosthetic docking device and comprising a plurality of interconnected struts and a plurality of projections extending from the plurality of interconnected struts. The prosthetic docking device and the anchoring frame are configured to be moved from a radially compressed configuration to a radially expanded configuration. The projections of the anchoring frame are configured to engage native tissue at an implantation location to help prevent the assembly from moving relative to the implantation location.

In another representative example, an assembly comprises a prosthetic device, a first anchoring frame, and a second anchoring frame. The prosthetic docking device including a frame configured for receiving and supporting a prosthesis therein. The frame comprises a first end portion and a second end portion. The first anchoring frame is coupled to first end portion of the frame of the prosthetic docking device and comprises a first plurality of interconnected struts and a first plurality of projections extending from the first plurality of interconnected struts. The second anchoring frame is coupled to the frame of the prosthetic docking device and comprises a second plurality of interconnected struts and a second plurality of projections extending from the second plurality of interconnected struts.

In another representative example, an assembly comprises a prosthetic heart valve, a first anchoring frame, and a second frame. The prosthetic heart valve including a frame and a valvular structure supported within the frame. The frame comprises a first end portion and a second end portion. The first anchoring frame coupled to first end portion of the frame of the prosthetic heart valve and comprising a first plurality of interconnected struts and a first plurality of projections extending from the first plurality of interconnected struts. The second anchoring frame coupled to the frame of the prosthetic heart valve and comprising a second plurality of interconnected struts and a second plurality of projections extending from the second plurality of interconnected struts.

In another representative example, an assembly comprises a graft, a first anchoring frame, and a second anchoring frame. The graft including a frame that is radially expandable within a blood vessel. The first anchoring frame coupled to first end portion of the frame of the graft and comprising a first plurality of interconnected struts and a first plurality of projections extending from the first plurality of interconnected struts. The second anchoring frame coupled to the frame of the graft and comprising a second plurality of interconnected struts and a second plurality of projections extending from the second plurality of interconnected struts.

In another representative example, an assembly comprises a graft and an anchoring frame. The graft including a frame that is radially expandable within a blood vessel. The anchoring frame coupled to the frame of the graft and comprising a plurality of interconnected struts and a plurality of projections extending from the plurality of interconnected struts. The graft and the anchoring frame are configured to be moved from a radially compressed configuration to a radially expanded configuration. The projections of the anchoring frame are configured to engage native tissue at an implantation location to help prevent the assembly from moving relative to the implantation location.

In another representative example, a prosthetic heart valve assembly comprises a prosthetic heart valve, an anchoring frame, and one or more sutures. The prosthetic heart valve comprising a valve frame and a valvular structure. The valve frame comprises a plurality of struts. The valve frame is expandable from a radially compressed delivery configuration to a radially expanded configuration. The valvular structure comprising a plurality of leaflets. The valvular structure is disposed radially within and is coupled to the valve frame. The valvular structure is configured to allow blood flow through the prosthetic heart valve assembly in a first direction and to restrict blood flow through the prosthetic heart valve assembly in a second direction. The anchoring frame comprising a plurality of struts. The anchoring frame is disposed radially outwardly from and is coupled to the valve frame. The struts of the anchoring frame comprise one or more tissue-engaging elements configured for contacting native tissue to help secure the prosthetic heart valve assembly at an implantation location. The one or more sutures extend circumferentially around the anchoring frame and configured for reducing paravalvular leakage between the native tissue and the prosthetic heart valve.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 depicts a perspective view of an exemplary prosthetic heart valve assembly.

FIG. 2 depicts a detail view of the prosthetic heart valve assembly of FIG. 1.

FIG. 3A depicts a perspective view of a valve frame of the prosthetic heart valve assembly of FIG. 1, depicting the valve frame in an annular configuration.

FIG. 3B depicts a perspective view of an anchoring frame of the prosthetic heart valve assembly of FIG. 1, depicting the anchoring frame in an annular configuration.

FIG. 4A depicts a side elevation view of the valve frame of the prosthetic heart valve assembly of FIG. 1, depicting the valve frame in a flat configuration.

FIG. 4B depicts a side elevation view of the anchoring frame of the prosthetic heart valve assembly of FIG. 1, depicting the anchoring frame in a flat configuration.

FIG. 5A depicts a cross-sectional view of a strut of the valve frame, taken along the line 5A-5A depicted in FIG. 3A.

FIG. 5B depicts a cross-sectional view of a strut of the anchoring frame, taken along the line 5B-5B depicted in FIG. 3B.

FIGS. 6-11 depict various views of the prosthetic heart valve assembly being delivered and implanted in a native aortic valve of a heart, which is shown in partial cross-section.

FIG. 12 depicts another exemplary prosthetic heart valve assembly implanted in a native mitral valve of a heart, which is shown in partial cross-section.

FIG. 13 depicts a perspective view of another exemplary prosthetic heart valve assembly.

FIG. 14 depicts a perspective view of another exemplary prosthetic heart valve assembly.

FIG. 15 depicts a perspective view of another exemplary prosthetic heart valve assembly.

FIG. 16 depicts a partial side elevation view of another anchoring frame for a prosthetic heart valve assembly.

FIG. 17 depicts a detail view of the anchoring frame of FIG. 16.

FIG. 18 depicts a side elevation view of another anchoring frame for a prosthetic heart valve assembly, depicting the anchoring frame in a flat configuration.

FIG. 19 depicts a detail view of the anchoring frame of FIG. 18.

FIG. 20A depicts a detail view of the anchoring frame of FIG. 18, depicting the anchoring frame in a radially compressed configuration.

FIG. 20B depicts a detail view of the anchoring frame of FIG. 18, depicting the anchoring frame in a radially expanded configuration.

FIG. 21A depicts a detail view of the anchoring frame of FIG. 18, depicting the anchoring frame in the radially compressed configuration and native leaflet tissue adjacent to the anchoring frame.

FIG. 21B depicts a detail view of the anchoring frame of FIG. 18, depicting the anchoring frame in the radially expanded configuration and native leaflet tissue captured between tines of the anchoring frame.

FIG. 22 depicts a side elevation view of another anchoring frame, depicting the anchoring frame in a radially expanded configuration.

FIGS. 23A-23C schematically depict partial side views of the anchoring frame of FIG. 22, depicting tines of the anchoring frame in various configurations.

FIGS. 24A-24C schematically depict other partial side views of the anchoring frame of FIG. 22, depicting the tines of the anchoring frame in various configurations.

FIG. 25 depicts a side elevation view of the anchoring frame of FIG. 22, depicting the anchoring frame in a radially compressed configuration.

FIG. 26 depicts a side elevation view of another anchoring frame, depicting the anchoring frame in a radially compressed configuration.

FIG. 27 depicts a perspective view of another prosthetic heart valve assembly.

FIG. 28 depicts a detail view of a frame and friction-increasing elements of the prosthetic heart valve assembly of FIG. 27.

FIG. 29 depicts a perspective view of another prosthetic heart valve assembly.

FIG. 30 depicts a perspective view of a frame for a prosthetic heart valve assembly, depicting the frame in a radially expanded configuration.

FIG. 31 depicts a detail view of the frame of FIG. 30.

FIG. 32 depicts a perspective view of the frame of FIG. 30, depicting the frame in a radially compressed configuration.

FIG. 33A depicts a perspective view of another anchoring frame, the anchoring frame having a relatively short axial length (e.g., compared to the anchoring frame of FIG. 34).

FIG. 33B depicts a detail view of the anchoring frame of FIG. 33A.

FIG. 34 depicts a perspective view of another anchoring frame, the anchoring frame having a relatively long axial length (e.g., compared to the anchoring frame of FIG. 33A).

FIG. 35 depicts a perspective view of another frame for a prosthetic heart valve including a sealing member, according to one example.

FIG. 36A depicts a perspective view of an expandable suture in a tensioned configuration.

FIG. 36B depicts a perspective view of the expandable suture of FIG. 36A in a relaxed or puffy configuration.

### DETAILED DESCRIPTION

General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

Introduction to the Disclosed Technology

The native valves of the heart can suffer from various problems that cause the native valve to improperly function. For example, aortic insufficiency (“AI”) or aortic regurgitation (“AR”) is characterized by diastolic reflux of blood through the native aortic valve back into the left ventricle (“LV”). Chronic AI can result in LV volume increase and/or pressure overload due to the heart's attempt to overcome the reduced cardiac output.

These conditions can, in some instances, be treated by implanting a prosthetic heart valve to replace the functionality of the malfunctioning native valve. Some prosthetic heart valves can be implanted in a minimally-invasive manner. These valves are typically referred to as “transcatheter heart valves” (“THVs”). THVs have many advantages, including requiring less recovery time and/or being suitable for a wider range of patients. Despite these advantages, many known THVs are designed to be deployed in a native heart valve annulus (e.g., a native aortic valve annulus) in cases of native stenosis or calcification of the native leaflets. In the absence of stenosis or calcification, many typical THVs lack sufficient anchoring mechanisms to secure the THV relative to the native anatomy. This can, in some instances, cause the THV to migrate and/or slip out of position under physiological pressures at the implantation site.

Thus, it is desirable to provide a prosthetic heart valve that can be anchored against the native anatomy in the absence of local stenosis and/or calcified anatomy. Additionally, it is desirable that the proposed solution can be used with existing prosthetic heart valves to eliminate the need for complex and time-intensive redesign.

Described herein are prosthetic heart valves and methods for implanting prosthetic heart valves. In some examples, a prosthetic heart valve assembly is disclosed. The disclosed prosthetic heart valve assemblies have anchoring frames and/or other features coupled to the valve frame that are configured for securing the prosthetic heart valves to the native tissue. The anchoring frames can, in some instances, comprise tissue-engaging elements configured to increase friction between the prosthetic heart valve assembly and the native tissue. As such, the prosthetic heart valve assemblies disclosed herein can resist migration relative to the native tissue. The disclosed assemblies can be used, for example, in patients with relatively non-stenotic anatomy and/or at implantation locations that do not provide sufficient structure for a typical prosthetic heart valve (e.g., at a native mitral valve). Additionally, the anchoring frames and/or other tissue-engaging elements disclosed herein can, for example, be attached (e.g., retrofitted) to a prosthetic heart valve. Therefore, the disclosed devices and methods improve stability and reduce migration of prosthetic heart valves, while also being relatively simple and cost-effective to implement.

In particular examples, the disclosed prosthetic heart valve assemblies can retain their positioning relative to a native aortic annulus under blood pressure as high as 250 mmHg (average blood pressure is 100 mmHg).

In lieu of or in addition to the various native valves of a heart, the prosthetic valve assemblies disclosed herein can be configured to be implanted at other locations within a patient's vasculature, such as a blood vessel (e.g., a vena cava).

Also disclosed herein are frames for various other types of devices (i.e., besides prosthetic heart valve frames). These frames can be coupled to or integrally formed with valve docking devices, grafts, stents, and/or other devices that are implanted within and engage a patient's vasculature (e.g., heart valve tissue, blood vessels, etc.).

Described below are some particular examples of the disclosed technology.

Examples of the Disclosed Technology

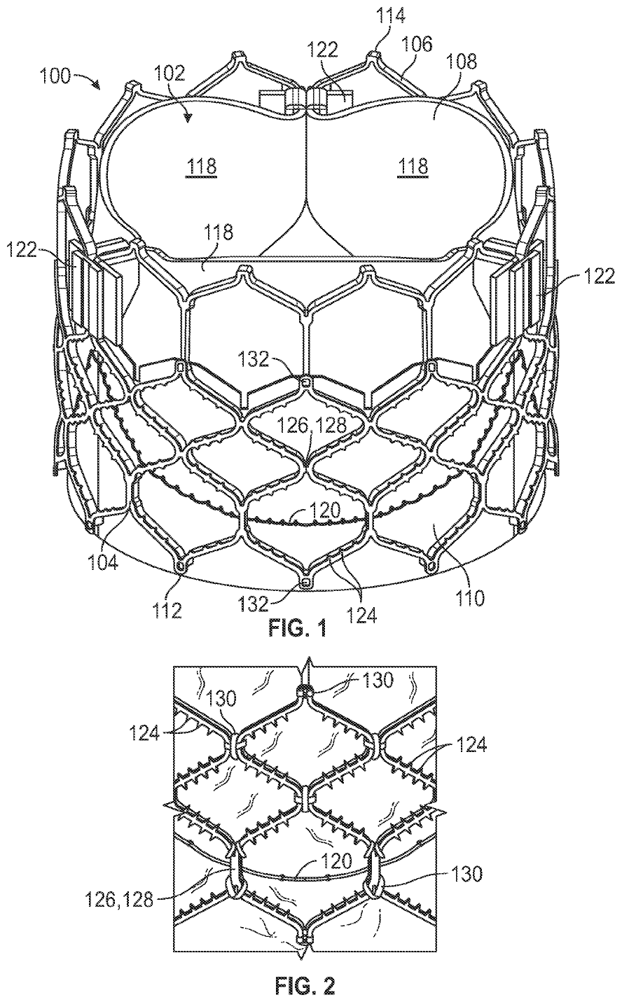

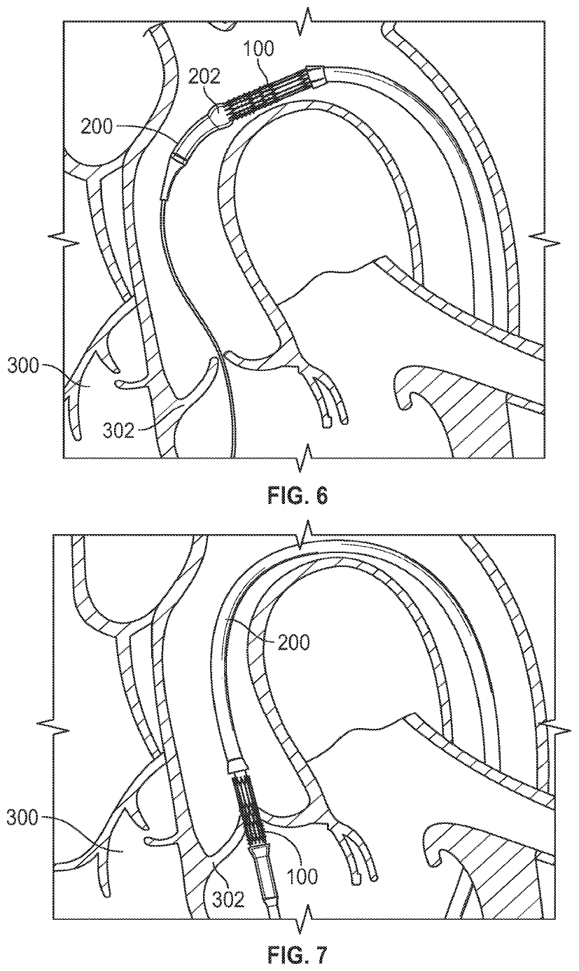

FIGS. 1-5B depict a prosthetic heart valve assembly 100 or its components. FIGS. 6-11 depict the prosthetic heart valve assembly 100 being implanted in a native aortic valve of a heart via an exemplary implantation method.

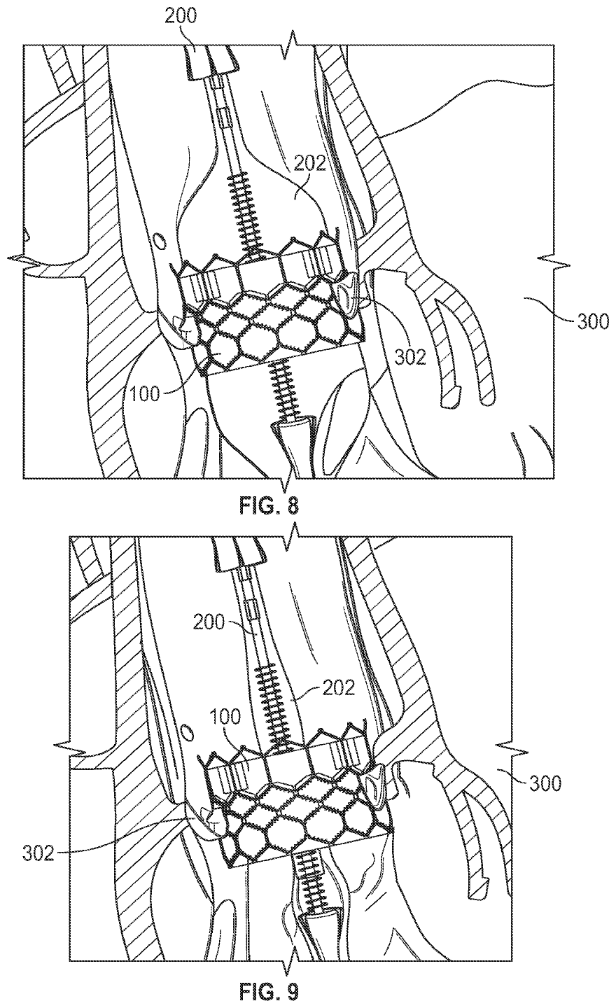

Referring to FIG. 1, the prosthetic heart valve assembly 100 comprises a prosthetic heart valve 102 and an anchoring frame 104 coupled to a valve frame 106 of the prosthetic heart valve 102. Due to their relative locations, the anchoring frame 104 can also be referred to as “the outer frame,” and the valve frame 106 can also be referred to as “the inner frame.” The prosthetic heart valve assembly 100 can be radially compressed (which can also be referred to as “crimped”) to a delivery configuration (e.g., FIGS. 6-7) and advanced through a patient's vasculature to an implantation location. The prosthetic heart valve assembly 100 can be radially expanded from the delivery configuration to a functional configuration and positioned in a native heart valve annulus (e.g., FIGS. 8-10). The prosthetic heart valve 102 is configured for regulating the flow of blood in one direction through the prosthetic heart valve assembly 100, and the anchoring frame 104 comprises a plurality of tissue-engagement elements (e.g., the projections 124) configured to help secure the prosthetic heart valve 102 to native heart valve tissue and/or to help promote tissue ingrowth between the native tissue and the prosthetic heart valve assembly 100.

As an overview and referring again to FIG. 1, the prosthetic heart valve 102 comprises the valve frame 106, a valvular structure 108, and optionally one or more sealing members 110 (which can also be referred to as “a sealing skirt” or “a PVL skirt”). The valve frame 106 is configured for supporting the valvular structure 108 and/or to help secure the prosthetic heart valve assembly 100 to native heart valve tissue (e.g., a native heart valve annulus and/or native leaflets). The valvular structure 108 is configured to open to allow blood flow through the prosthetic heart valve 102 from an inflow end portion 112 to an outflow end portion 114. The valvular structure 108 is also configured to close to prevent or restrict blood flow through the prosthetic heart valve 102 from the outflow end portion 114 to the inflow end portion 112. The sealing member 110 is configured for reducing or eliminating blood flow around the valvular structure 108 and/or native tissue (which can also be referred to as “paravalvular leakage,” “perivalvular leakage”, or “PVL”).

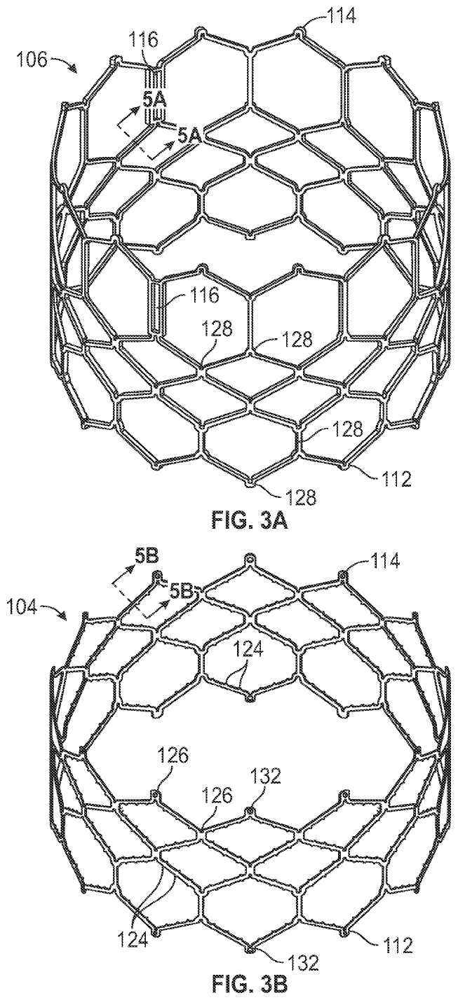

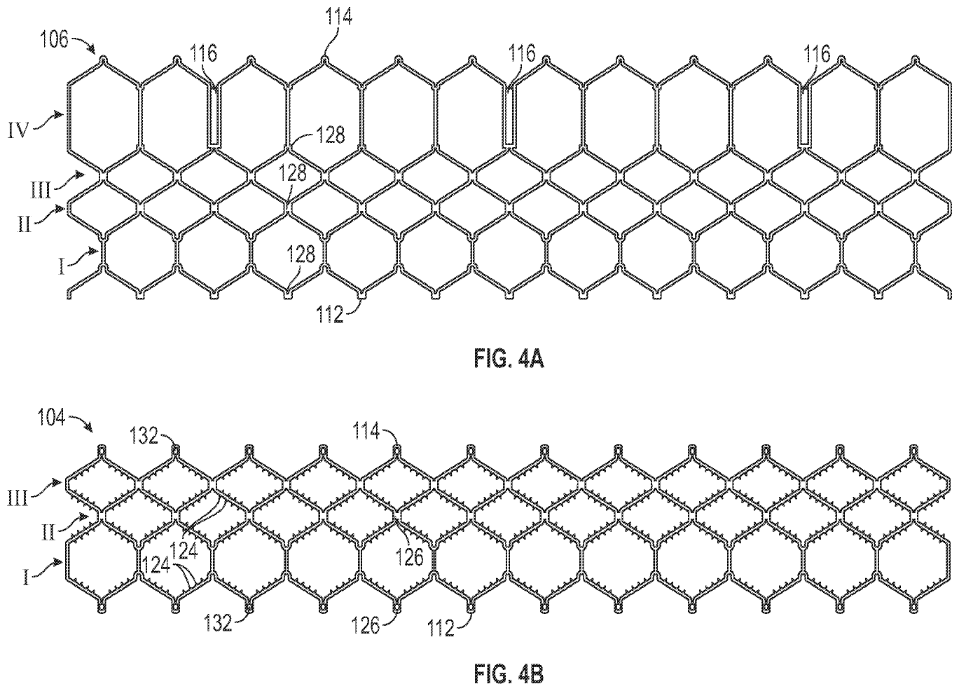

FIGS. 3A and 4A depict the valve frame 106 of the prosthetic heart valve 102 with the other components removed. FIG. 3A depicts the valve frame 106 in an annular configuration, which corresponds to its functional configuration, and FIG. 4A depicts the valve frame 106 in a flat configuration for purposes of illustration. The valve frame 106 comprises a plurality of interconnected struts. In some examples, the struts form a plurality of cells. For example, referring to FIG. 4A, the struts of the valve frame 106 form a plurality of rows of closed cells, including a first row of closed cells I, a second row of closed cells II, a third row of closed cells III, and a fourth row of closed cells IV. In the illustrated example, the cells of row I are larger than the cells of rows II and III but are smaller than the cells of row IV. The cells of row II are the same size or at least substantially the same size as the cells of row III. In the depicted example, the cells are generally hexagonal shaped. In other examples, a valve frame can comprise various other numbers of rows of cells, the cells can comprise different sizes, and/or the cells can comprise different shapes.

The valve frame 106 also comprises a plurality of commissure windows 116 (e.g., three in the illustrated example). The commissure windows 116 are configured for coupling the valvular structure 108 to the valve frame 106.

The valve frame can be made of any of various suitable plastically-expandable materials (e.g., stainless steel, etc.) and/or self-expanding materials (e.g., Nitinol). When the valve frame comprises plastically-expandable material, the valve frame (and thus prosthetic heart valve assembly) can be crimped to a radially compressed state on a delivery catheter and then expanded inside a patient by an inflatable balloon or equivalent expansion mechanism of a delivery apparatus. When the valve frame comprises self-expandable material, the valve frame (and thus the prosthetic heart valve assembly) can be crimped to a radially compressed state and restrained in the compressed state by a sheath or equivalent mechanism of a delivery apparatus. Once inside the body, the prosthetic heart valve assembly can be advanced from the delivery sheath, which allows the valve to self-expand to its functional size.

Suitable plastically-expandable materials that can be used to form the valve frame include stainless steel, nickel-based alloys (e.g., cobalt-chromium alloy or nickel-cobalt-chromium alloy), polymers, and/or combinations thereof. In particular examples, the valve frame is made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details about valve frames can be found in U.S. Pat. No. 9,393,110 and U.S. Publication No. 2018/0028310, both of which are incorporated by reference herein.

Referring again to FIG. 1, the valvular structure 108 comprises a plurality of leaflets 118 (e.g., three leaflets in the illustrated example), which collectively form a leaflet structure. The lower edge of the leaflet structure has an undulating, curved scalloped shape. The suture line 120 generally tracks the scalloped shape of the leaflet structure. The upper edge of each leaflet 118 comprises a tab 122. The tabs 122 of adjacent pairs of leaflets can be joined together to form commissures. The tabs 122 can be inserted through the commissure windows 116 of the valve frame 106 and secured to the valve frame 106.

Additional details about valve structure and the manner in which a valve structure can be secured to a valve frame can be found in U.S. Pat. No. 9,393,110 and U.S. Publication No. 2018/0028310.

The leaflets 118 can be formed of pericardial tissue (e.g., bovine, porcine, and/or equine pericardial tissue), biocompatible synthetic materials, and/or various other suitable natural or synthetic materials as described in U.S. Pat. No. 6,730,118, which is incorporated by reference herein.

The sealing member 110 can assist in securing the valvular structure 108 to the valve frame 106 and in forming a good seal between the prosthetic heart valve assembly 100 and the native annulus by blocking the flow of blood through the open cells of the valve frame 106 below the lower edge of the leaflets 118. In the illustrated example, the sealing member 110 is disposed on the inside of valve frame 106. In other examples, the sealing member can extend from the inside of the valve frame to the outside of the valve frame. Additionally or alternatively, the prosthetic heart valve 102 can comprise a plurality of sealing members, including a first sealing member (i.e., an inner skirt) disposed on the inside of the valve frame and a second sealing member (i.e., an outer skirt) disposed on the outside of the valve frame.

The sealing member 110 can be formed of various materials such as a fabric or cloth. In some instances, the sealing member can be formed from polyethylene terephthalate (“PET”) and/or ultra-high molecular weight polyethylene (“UHMWPE”) fabric. In other examples, various other synthetic or natural materials can be used.

The valvular structure 108 can be attached to the sealing member 110 in various ways, including sutures, fasteners, etc. Additional details about sealing members can be found in U.S. Pat. No. 9,393,110 and U.S. Publication No. 2018/0028310.

Referring to FIG. 3B, the anchoring frame 104 comprises a plurality of struts configured in an annular shape. As depicted in FIG. 2, the anchoring frame 104 also comprises a plurality of tissue-engaging elements. In the illustrated example, the tissue-engaging elements are projections 124 (which can also be referred to as “anchors”) extending from the struts of the anchoring frame 104. The projections 124 are configured to engage (and in some instances penetrate) the native heart valve tissue. In this manner, the projections 124 can increase the frictional engagement between the prosthetic heart valve assembly 100 and native heart valve tissue, which can help to reduce migration of the prosthetic heart valve assembly 100 relative to the native heart valve tissue after it is released from the delivery apparatus. The projections can also help to improve tissue ingrowth and/or reduce PVL.

The projections 124 can extend in various directions from the struts of the anchoring frame 104. For example, in the illustrated example, some of the projections 124 extend from the struts at an angle relative to a central longitudinal axis extending from the inflow end to the outflow end of the prosthetic heart valve assembly. In some instances, the projections are perpendicular or at least substantially perpendicular (e.g., forming an angle of 80-100 degrees) to the struts from which they extend. In other examples, the projections can extend from their respective struts at various other angles (e.g., between 1-79 degrees). For example, in some instances, the projections can extend from their respective struts at an angle of about 45 degrees such that projections are parallel or at least substantially parallel to a central longitudinal axis extending from the inflow end to the outflow end of the prosthetic heart valve assembly.

The projections can comprise various shapes and lengths such that the projections provide sufficient retention force for the prosthetic heart valve assembly, while reducing potential harm to the surrounding tissue. For example, in the illustrated example, the projections comprise tines or spikes. In other examples, the projections can comprise ball-shaped bulges and/or a rectangular shape. Additionally or alternatively, one or more of the projections can comprise a curved shape, a hook shape, a cross shape, a T-shape, and/or a barbed shape. Various combinations of shapes and/or sizes of projections can be used.

The anchoring frame 104 of the prosthetic heart valve assembly 100 can be formed as a separate component that is attached to the prosthetic heart valve 102 to form the assembly. FIGS. 3B and 4B depict the anchoring frame 104 of the prosthetic heart valve assembly 100 detached from the prosthetic heart valve 102. FIG. 3B depicts the anchoring frame 104 in an annular configuration, and FIG. 4B depicts the anchoring frame 104 in a flat configuration.

In some examples, the struts of the anchoring frame 104 can form a plurality of cells. For example, referring to FIG. 4B (which depicts the anchoring frame 104 in a flat configuration), the struts of anchoring frame 104 form a plurality of rows of closed cells, including a first row of closed cells I, a second row of closed cells II, and a third row of closed cells III. In the illustrated example, the cells of the first row I are larger than the cells of rows II and III. The cells of the second row II are the same size or at least substantially the same size as the cells of the third row III. The cells are generally hexagonal shaped. In other examples, an anchoring frame can comprise various other numbers of rows of cells (e.g., 1, 2, 4), the cells can comprise different sizes, and/or the cells can comprise different shapes.

The anchoring frame 104 is configured such that the struts and the cells of the rows I, II, and III of the anchoring frame 104 align with the struts and the cells of the rows I, II, and III of the valve frame 106, respectively (see, e.g., FIG. 1). In this manner, strut junctions 126 of the anchoring frame 104 can be coupled to strut junctions 128 of valve frame 106, as depicted in FIG. 2. In the illustrated example, the anchoring frame 104 is coupled to the valve frame 106 with sutures 130. In some examples, the anchoring frame 104 comprises openings 132, which can be configured to receive the sutures 130. It should be noted that, for purposes of illustration, the sutures are not shown in FIG. 1. In other examples, the anchoring frame can be coupled to the valve frame in various other ways (e.g., fasteners, welding, adhesive, etc.). By coupling the junctions 126 of the anchoring frame 104 to junctions 128 of the valve frame 106, the anchoring frame 104 can, for example, expand and/or or compress simultaneously with the valve frame 106.

In some examples, the anchoring frame 104 is removably coupled to the valve frame 106 (e.g., with the sutures 130 and/or fasteners). As used herein, “removably coupled” means coupled in such a way that two components are coupled together and can be separated without plastically deforming either of the components. In other examples, the anchoring frame can be permanently coupled to the valve frame (e.g., via welding). As used herein, “permanently coupled” means coupled in such a way that the two components cannot be separated without plastically deforming at least one of the components.

The anchoring frame can be made of any of various suitable plastically-expandable materials (e.g., stainless steel, etc.) and/or self-expanding materials (e.g., Nitinol). When the anchoring frame comprises plastically-expandable material, the anchoring frame (and thus prosthetic heart valve assembly) can be crimped to a radially compressed state on a delivery catheter and then expanded inside a patient by an inflatable balloon or equivalent expansion mechanism of a delivery apparatus. When the anchoring frame comprises self-expandable material, the anchoring frame (and thus the prosthetic heart valve assembly) can be crimped to a radially compressed state and restrained in the compressed state by a sheath or equivalent mechanism of a delivery apparatus. Once inside the body, the prosthetic heart valve assembly can be advanced from the delivery sheath, which allows the prosthetic heart valve assembly to expand to its functional size.

Suitable plastically-expandable materials that can be used to form the anchoring frame include stainless steel, nickel based alloys (e.g., cobalt-chromium alloy or nickel-cobalt-chromium alloy), polymers, and/or combinations thereof In particular examples, the anchoring frame is made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

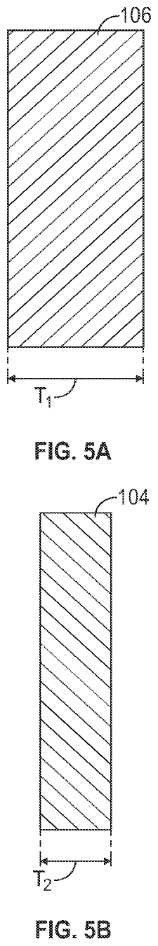

The thicknesses of the anchoring frame 104 and the valve frame 106 are depicted schematically in FIGS. 5A-5B. FIG. 5A depicts a cross-sectional view of a strut of the valve frame 106. FIG. 5B depicts a cross-sectional view of a strut of the anchoring frame 104. The valve frame 106 comprises a thickness T₁, as shown in FIG. 5A. The anchoring frame 104 comprises a thickness T₂, as shown in FIG. 5B. The anchoring frame 104 can be relatively thinner than the valve frame 106 because the anchoring frame 104 does not support loads applied to the valve frame by the valvular structure 108 (at least not substantially). In some examples, the thickness T₁ of the valve frame 106 is within a range of 0.3-0.9 mm and the thickness T₂ of the anchoring frame 104 is within a range of 0.01-0.1 mm. In some examples, a ratio of the thickness T₂ of the anchoring frame 104 to the thickness T₁ of the valve frame 106 can be within a range of 0.3-0.9. In instances when the frames do not have uniform thicknesses (or substantially uniform thicknesses), the thicknesses T₁ and T₂ can be the nominal or the average thicknesses of the frames.

Since the anchoring frame is relatively thin and flexible, it does not significantly increase the radial profile of the prosthetic heart valve in the radially compressed configuration. This can, for example, allow the prosthetic heart valve assembly 100 to be implanted in the same manner as a prosthetic heart valve that does not include the anchoring frame 104. It can also allow the same delivery apparatus to be used for delivering the prosthetic heart valve 102 either with or without the anchoring frame 104 coupled thereto.

Thus, the anchoring frame 104 can improve the frictional engagement with the native heart valve tissue, while also being relatively easily coupled to a prosthetic heart valve. Therefore, the anchoring frame 104 provides improved anchoring without requiring that the prosthetic heart valve 102 and/or the delivery apparatus be modified to accommodate the anchoring frame 104.

As depicted in FIGS. 6-11, the prosthetic heart valve assembly 100 can be coupled to a delivery apparatus 200, which can be used to deliver, position, and secure the prosthetic heart valve assembly 100 in a native heart valve annulus. In the depicted implantation procedure, the prosthetic heart valve assembly 100 is implanted in a native aortic annulus of a heart using a transfemoral delivery approach. In other examples, the prosthetic heart valve assembly 100 can be implanted at other locations (e.g., a mitral valve, a tricuspid valve, and/or a pulmonary valve) and/or using other delivery approaches (e.g., transapical, transaortic, transseptal, etc.).

The prosthetic heart valve assembly can be releasably coupled to a distal end portion of a delivery apparatus by positioning the prosthetic heart valve assembly over an inflatable balloon disposed at a distal end portion of the delivery apparatus and radially compressing the prosthetic heart valve assembly to a delivery configuration. As depicted in FIG. 6, the distal end portion of the delivery apparatus 200, which comprises the balloon 202, and the radially compressed prosthetic heart valve assembly 100 can be inserted percutaneously into a patient's vasculature and advanced toward a heart 300. As shown in FIG. 7, the prosthetic heart valve assembly 100 can be disposed in or adjacent a native aortic annulus 302 of the heart 300. The balloon 202 can then be inflated to radially expand the prosthetic heart valve 102 from the radially compressed delivery configuration to a radially expanded, functional configuration, as shown in FIG. 8. The prosthetic heart valve assembly 100 can expand radially outwardly such that the anchoring frame 104 contacts native tissue of the heart 300 (e.g., the native aortic valve leaflets 304).

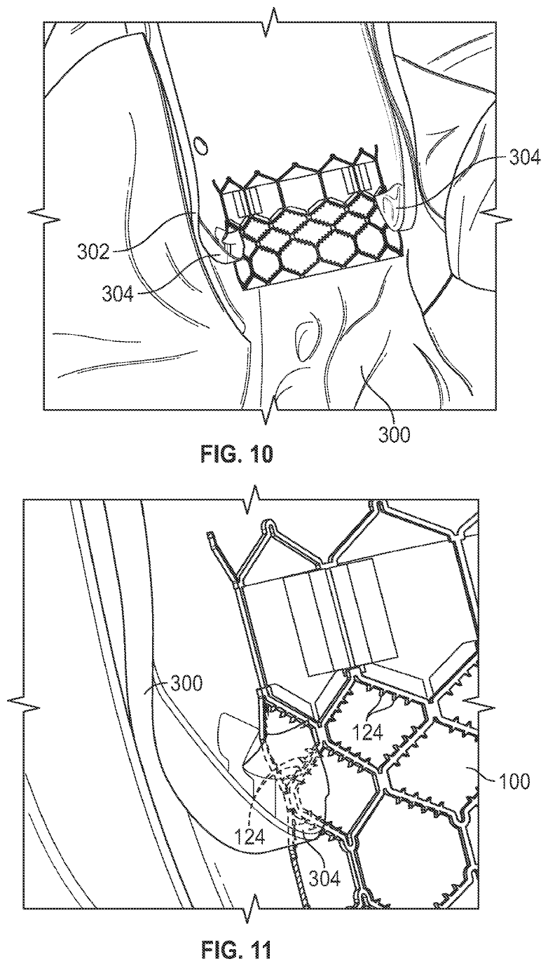

The projections 124 of the anchoring frame 104 can engage the native tissue, which can help to ensure that the prosthetic heart valve assembly 100 is secured relative to the native aortic annulus 302 of the heart 300. Once the prosthetic heart valve assembly 100 is secured within the native annulus, the balloon 202 of the delivery apparatus 200 can be deflated, as shown in FIG. 9. As depicted in FIG. 10, the delivery apparatus 200 can then be withdrawn from the patient's vasculature, leaving the prosthetic heart valve assembly 100 within the native aortic annulus 302 of the heart 300 to regulate blood flow from the left ventricle into the aorta. As shown in FIG. 11, the projections 124 of the anchoring frame 104 can (in some instances) penetrate the native tissue.

Because the anchoring frame 104 helps to secure the prosthetic heart valve assembly 100 relative to the native annulus, native tissue ingrowth with the prosthetic heart valve assembly 100 is improved. Also, the anchoring frame 104 reduces migration of the prosthetic heart valve assembly 100 relative to the native annulus, which allows the prosthetic heart valve assembly 100 to be implanted in a wider variety of patients. For example, the prosthetic heart valve assembly 100 can be implanted in patients with relatively less valvular calcification and/or or stenosis than is needed to anchor typical prosthetic heart valves.

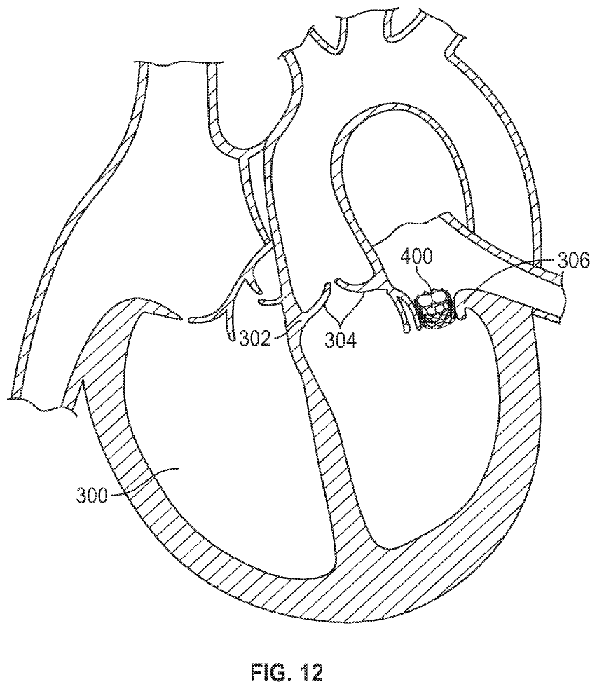

FIG. 12 depicts a prosthetic heart valve assembly 400 implanted in a native mitral valve 306 of the heart 300. The prosthetic heart valve assembly 400 can comprise a prosthetic heart valve and an anchoring frame configured similar to the prosthetic heart valve 102 and the anchoring frame 104, respectively. Although the prosthetic heart valve assembly 400 is generally similar to the prosthetic heart valve assembly 100, the prosthetic heart valve assembly 400 is configured for implantation in a native mitral valve. The prosthetic heart valve assemblies and/or the anchoring frames disclosed herein are particularly suitable for deployment in a native mitral valve, which can in some instances lack the sufficient anatomical structure to retain a typical prosthetic heart valve in place. This is because the disclosed prosthetic heart valve assemblies and/or anchoring frames provide increased frictional engagement with the native tissue, thereby improving the prosthetic heart valve assemblies' ability to resist migration relative to the native tissue, even in locations like the native mitral valve where typical prosthetic valves may be unsuitable.

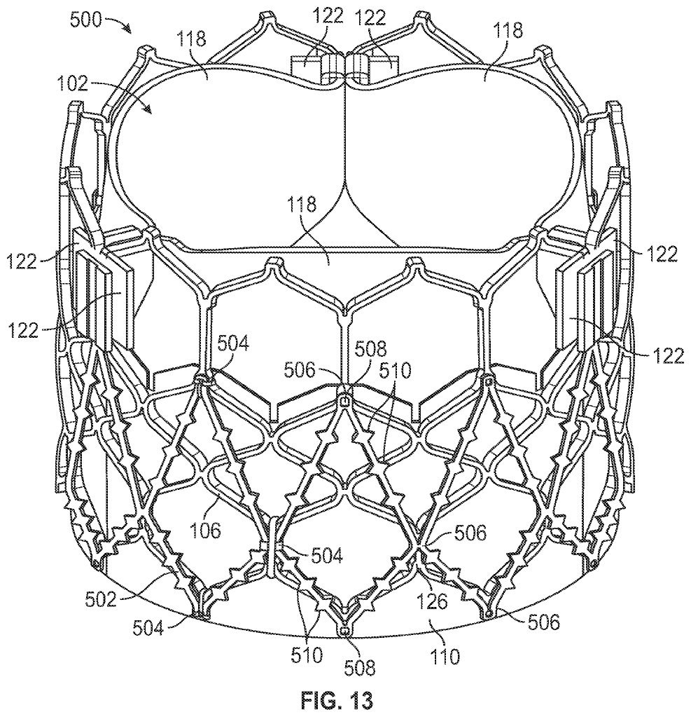

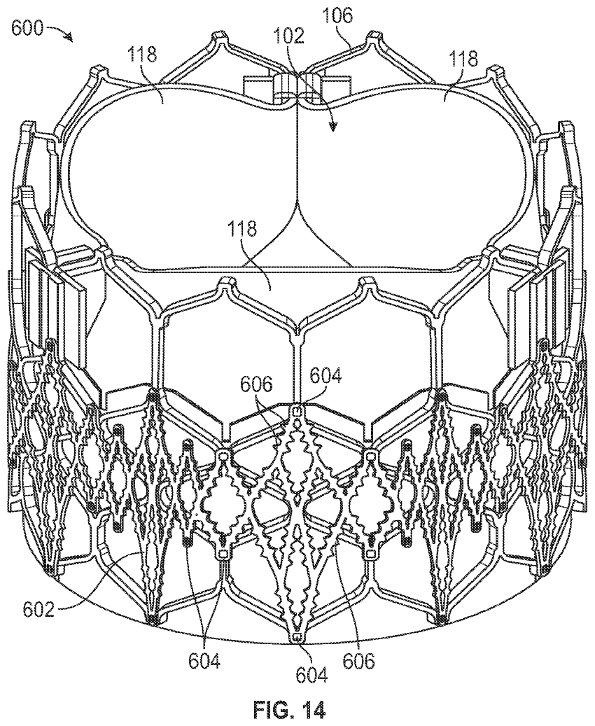

FIG. 13 depicts a prosthetic heart valve assembly 500, according to another example. The prosthetic heart valve assembly 500 comprises the prosthetic heart valve 102 and an anchoring frame 502 in lieu of the anchoring frame 104. The anchoring frame 502 is disposed radially outwardly from the valve frame 106. The anchoring frame 502 can be coupled to the valve frame 106 (e.g., with sutures 504) at junctions 506 of the struts of the anchoring frame 502 and junctions 126 of the valve frame 106. It should be noted that, for purposes of illustration, the sutures are not shown at every junction of the anchoring frame. To facilitate attaching the anchoring frame 502 to the valve frame 106, the anchoring frame 502 can, in some examples, comprise one or more openings 508 formed therein.

The anchoring frame 502 functions similar to the anchoring frame 104 in that it is configured to increase frictional engagement between the prosthetic heart valve assembly 500 and the native tissue adjacent to the prosthetic heart valve assembly. As such, the anchoring frame 502 can comprise one or more tissue-engaging elements 510. The tissue-engaging elements can comprise various shapes, sizes, and/or orientations.

Unlike the anchoring frame 104, however, the struts of the anchoring frame 502 do not align with the struts of the valve frame 106 (at least not to the same extent). The cells of the anchoring frame 502 are larger than the cells of the valve frame 106 with which they circumferentially overlap, and the struts of the anchoring frame 502 extend across one or more of the closed cells of the valve frame 106.

In the illustrated example, the anchoring frame 502 comprises a single row of closed cells. In other examples, an anchoring frame can comprise more than one row of closed cells (e.g., 2-12 rows of closed cells). In yet other examples, an anchoring frame may be formed without any rows of closed cells. For example, in some instances, an anchoring frame may have a plurality of interconnected struts that do not form closed cells (e.g., struts arranged in a zig-zag pattern).

Regardless of the particular strut configuration of the anchoring frame (e.g., open or closed cells, number of rows, etc.), the anchoring frame can be configured to expand and compress with the valve frame. This can be accomplished, for example, by aligning the junctions 506 (or nodes) of the anchoring frame 502 with the junctions 126 of the valve frame 106 and by coupling the anchoring frame to the valve frame.

FIG. 14 depicts a prosthetic heart valve assembly 600, according to another example. The prosthetic heart valve assembly 600 comprises the prosthetic heart valve 102 and an anchoring frame 602 in lieu of the anchoring frame 104. The anchoring frame 602 is disposed radially outwardly from the valve frame 106. The anchoring frame 602 can be coupled to the valve frame 106 (e.g., with sutures) at strut junctions of the anchoring frame 602 and strut junctions of the valve frame 106. It should be noted that, for purposes of illustration, the sutures are not shown. To facilitate attaching the anchoring frame 602 to the valve frame 106, the anchoring frame 602 can, in some examples, comprise one or more openings 604 formed therein. The sutures can extend through the openings 604 and/or around the struts of the anchoring frame 602 and the valve frame 106.

The anchoring frame 602 comprises three circumferentially-extending rows of cells. Compared to the cells of the anchoring frame 104 and the anchoring frame 502, cells of the anchoring frame 602 are relatively smaller.

The anchoring frame 602 is configured to increase frictional engagement between the prosthetic heart valve assembly 600 and the native tissue adjacent to the prosthetic heart valve assembly. As such, the anchoring frame 602 includes a plurality of tissue-engaging elements 606. The tissue-engaging elements can comprise various shapes, sizes, and/or orientations.

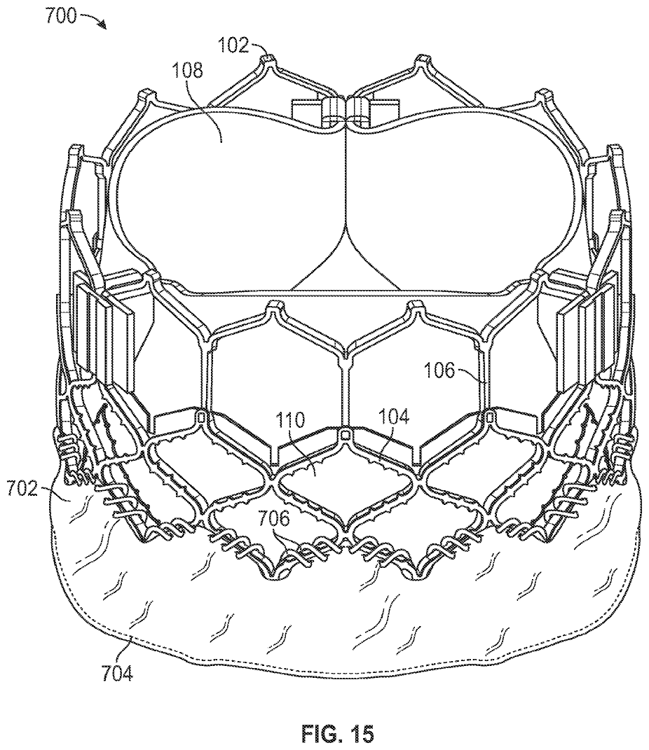

FIG. 15 depicts a prosthetic heart valve assembly 700. The prosthetic heart valve assembly 700 comprises the prosthetic heart valve 102, the anchoring frame 104, and an outer sealing member 702 (which can also be referred to as a “sealing skirt” or an “outer sealing skirt”). The outer sealing member 702 can be coupled to the inner sealing member 110 and to the anchoring frame 104 and/or the valve frame 106. The outer sealing member 702 is configured to reduce PVL.

The outer sealing member 702 can be coupled to the inner sealing member in various ways. For example, the outer sealing member 702 can be coupled to the inner sealing member 110 with stitching 704, as depicted in the illustrated example. In other examples, the outer sealing member can be coupled to the inner sealing member by integrally forming the inner and outer sealing members from a continuous piece of material (e.g., PET fabric).

The outer sealing member 702 can be coupled to the anchoring frame 104 and/or the valve frame 106 in various ways. For example, as depicted in FIG. 15, the outer sealing member 702 is coupled to the anchoring frame 104 and the valve frame 106 with sutures 706. In other examples, various other types of fasteners and/or other means for coupling can be used to couple the outer sealing member to the frames.

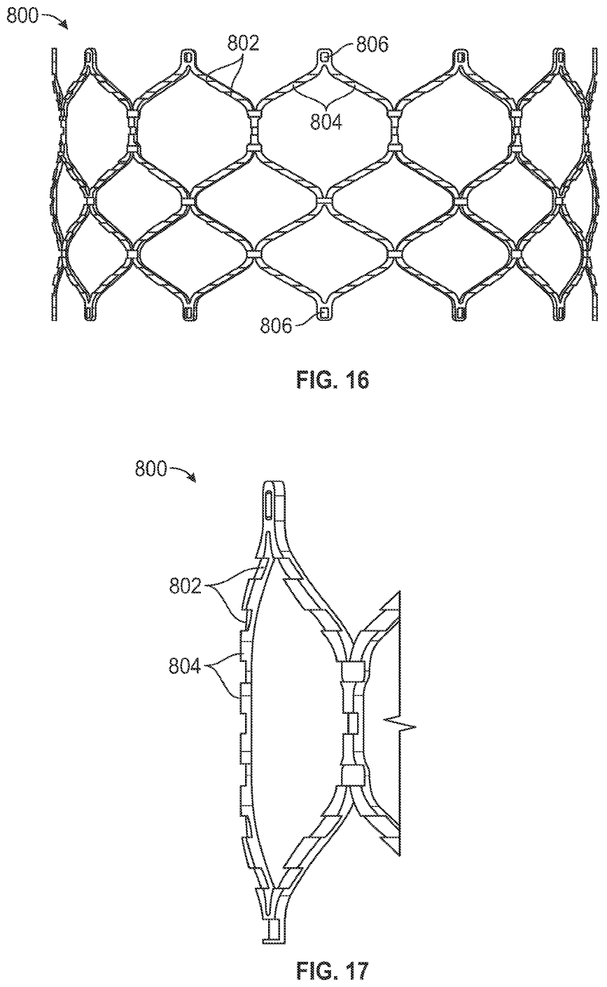

FIGS. 16-17 depict an anchoring frame 800, according to another example. The anchoring frame 800 can, for example, be coupled to a prosthetic heart valve (e.g., the prosthetic heart valve 102) to form a prosthetic heart valve assembly. The anchoring frame 800 is similar to the anchoring frame 104 in that the anchoring frame 800 is configured to increase frictional engagement between the prosthetic heart valve assembly and the native tissue adjacent to the prosthetic heart valve assembly. One difference between the anchoring frame 800 and the anchoring frame 104 is that the anchoring frame 800 comprises a plurality of grooves 802 formed in the radially outwardly facing surfaces of the struts of the anchoring frame 800 rather than having projections extending from the struts like the anchoring frame 104. The grooves 802 of the anchoring frame 800 are spaced apart so as to form ridges 804 between the grooves 802. The grooves 802 and the ridges 804 can engage native tissue and can increase friction compared to a relatively smooth frame as is found on typical prosthetic heart valves.

The anchoring frame 800 can also comprise one or more attachment features which can be used, for example, to couple the anchoring frame 800 to a prosthetic heart valve. In the illustrated example, the anchoring frame comprises a plurality of openings 806 disposed at apices of the anchoring frame. In lieu of or in addition to the openings at the apices, the anchoring frame 800 can comprise additional openings and/or other attachment features (e.g., at intermediate locations between the apices).

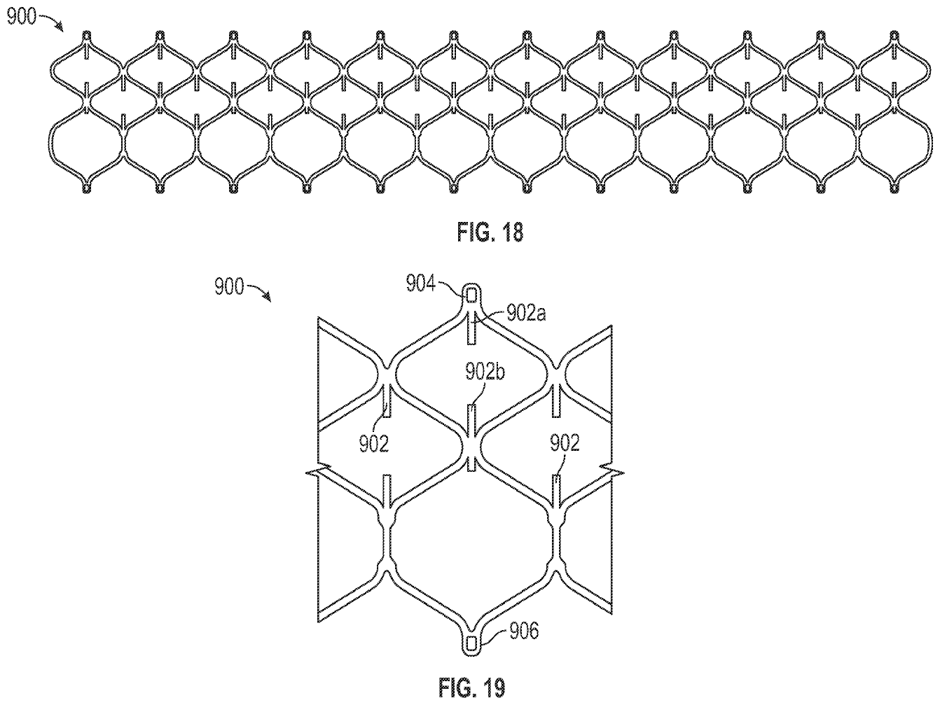

FIGS. 18-21B depict an anchoring frame 900, according to another example. Although FIG. 18 depicts the anchoring frame 900 in a flat configuration for purposes of illustration, the anchoring frame can comprise an annular configuration (e.g., similar to the annular configuration of the anchoring frame 104 depicted in FIG. 3B). The anchoring frame 900 can, for example, be coupled to a prosthetic heart valve (e.g., the prosthetic heart valve 102) to form a prosthetic heart valve assembly. The anchoring frame 900 is similar to the anchoring frame 104 in that the anchoring frame 900 is configured to increase frictional engagement between the prosthetic heart valve assembly and the native tissue adjacent to the prosthetic heart valve assembly.

The anchoring frame 900 comprises a plurality of tines 902 extending between junctions of the struts of the anchoring frame 900. Referring to FIG. 19, the tines 902 are arranged in pairs, with each tine of a pair being circumferentially aligned with each other and axially spaced apart from each other. For example, as depicted in FIG. 19, a pair of tines comprises a first tine 902a and a second tine 902b, which are circumferentially aligned and axially spaced. The first tine 902a is disposed toward a first end 904 of the anchoring frame 900 relative to the second tine 902b, and the first tine 902a extends axially toward the second tine 902b and a second end 906 of the anchoring frame 900. The second tine 902b is disposed toward the second end 906 of the anchoring frame 900 relative to the first tine 902a, and the second tine 902b extends axially toward the first tine 902a and the first end 904 of the anchoring frame 900.

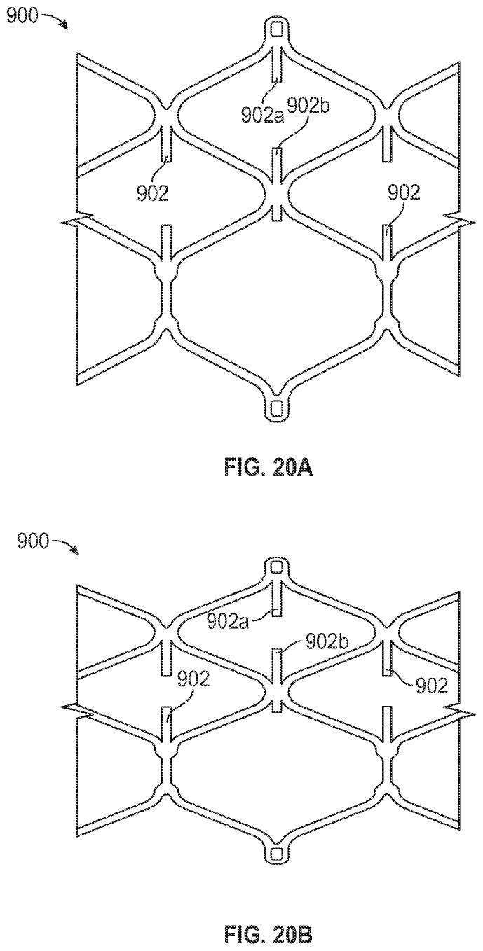

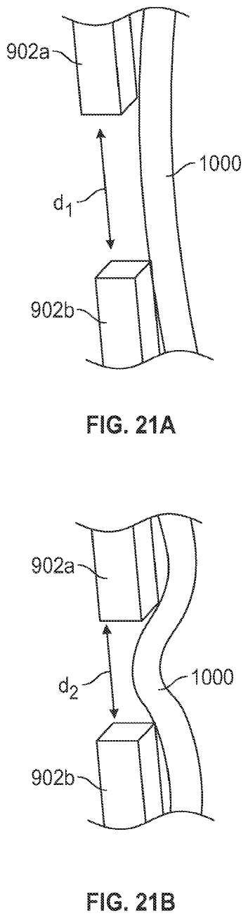

As depicted in FIG. 20A-20B, the anchoring frame 900 is configured to axially elongate when the anchoring frame is radially compressed (FIG. 20A) and to axially foreshorten when the anchoring frame is radially expanded (FIG. 20B). As the anchoring frame 900 axially elongates/radially compresses, each pair of tines moves away from each other. As the anchoring frame 900 axially foreshortens/radially expands, each pair of tines moves toward each other. Accordingly, when the anchoring frame 900 is radially expanded at an implantation location (e.g., a native heart valve annulus), the pairs of tines can, for example, capture (e.g., “pinch”) native tissue 1000 (e.g., a native leaflet) between the tines, as shown in FIGS. 21A-21B. In this manner, the anchoring frame 900 can help to ensure that a prosthetic heart valve to which the anchoring frame 900 is coupled is secured and does not migrate relative to the native tissue.

In some examples, one or more of the tines extends toward an inflow end of the anchoring frame, and one or more of the tines extends toward an outflow end of the anchoring frame. In certain examples, one or more of the tines is parallel or at least substantially parallel (e.g., within 1-10 degrees) to a central longitudinal axis of the anchoring frame. Additionally or alternatively, one or more of the tines can extend at an angle (e.g., 11-90 degrees) relative to the central longitudinal axis of the anchoring frame.

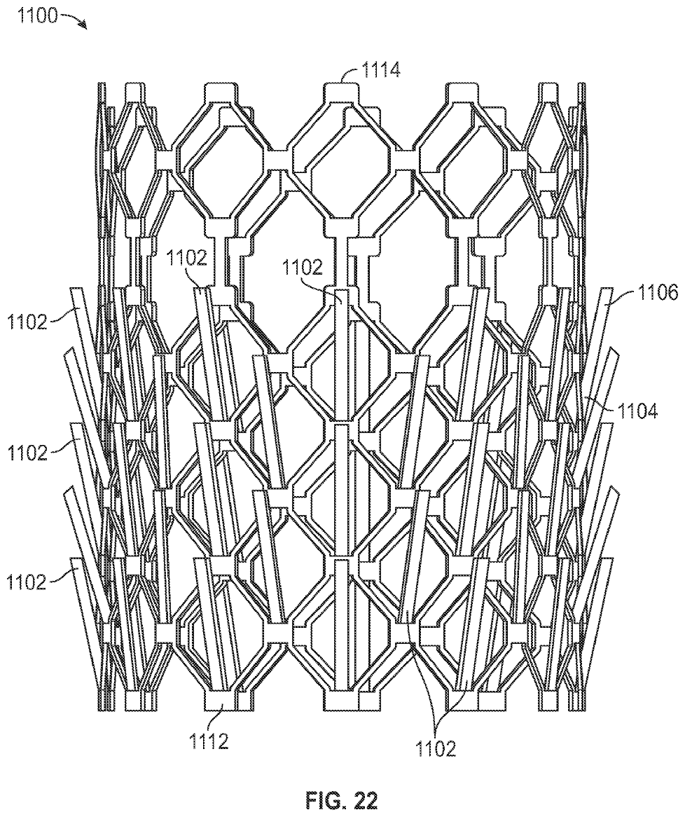

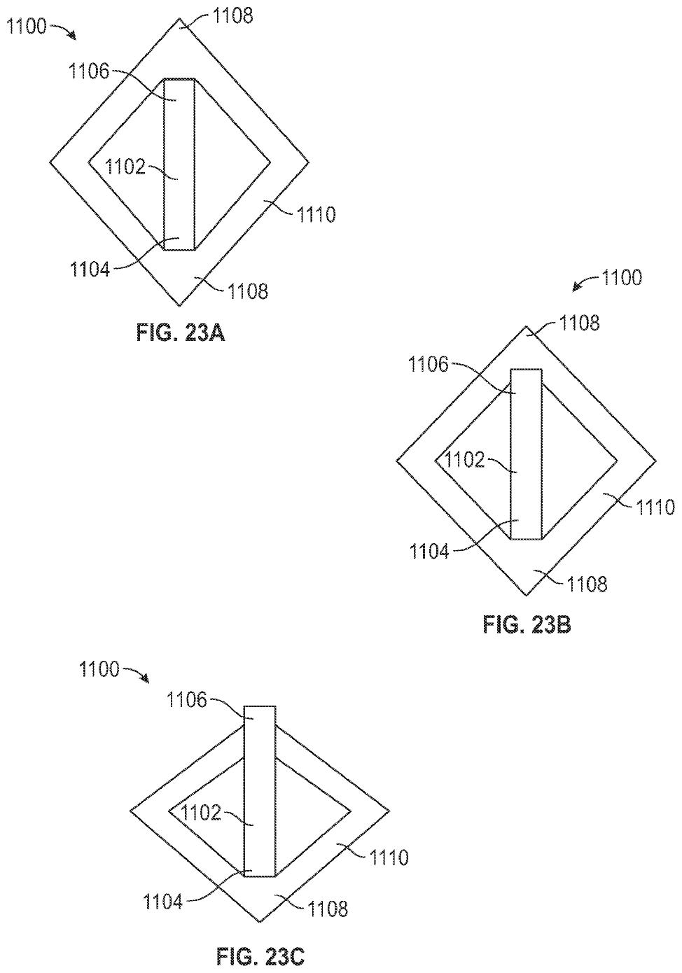

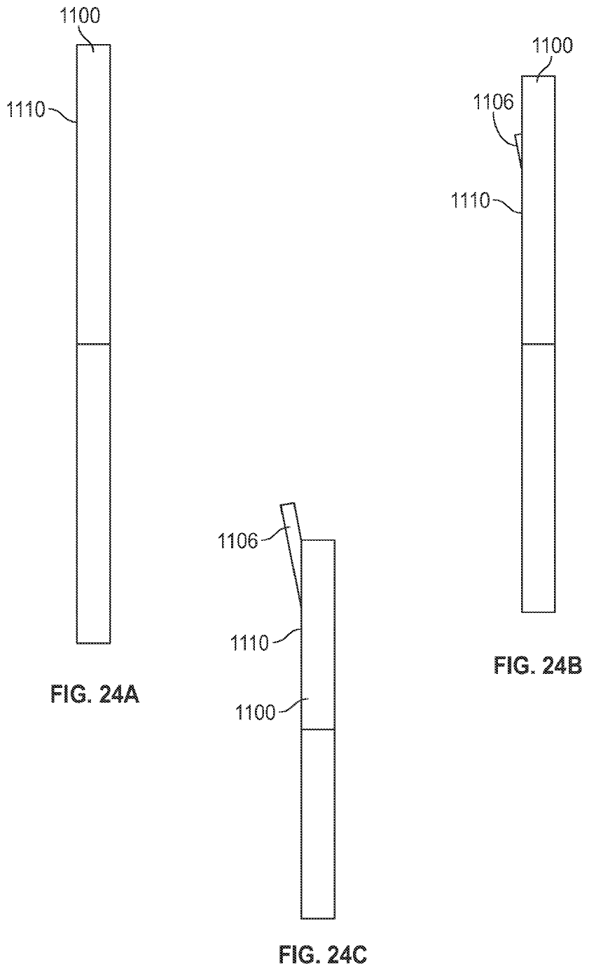

FIGS. 22-25 depict an anchoring frame 1100, according to another example. Referring to FIG. 22, the anchoring frame 1100 comprises a plurality of tines 1102 that are configured to engage native tissue (e.g., native leaflets). Referring to FIGS. 22 and 23C, each of the tines 1102 comprises a fixed end portion 1104 and a free end portion 1106. The fixed end portion 1104 can be coupled to the struts of the anchoring frame (e.g., at strut junctions 1108). The free end portion 1106 extends axially away from the fixed end portion 1104. As the anchoring frame 1100 radially expands and compresses, the free end portion 1106 can move axially relative to the struts of the anchoring frame 1100 (see, e.g., FIGS. 23A-23C) and can move radially relative to the struts of the anchoring frame 1100 (see, e.g., FIGS. 24A-24C).

The anchoring frame 1100 is configured such that the tines 1102 radially align with the other struts of the anchoring frame 1100 when the anchoring frame 1100 is in the radially compressed configuration and such that the tines 1102 extend radially outwardly from the other struts of the anchoring frame 1100 when the anchoring frame 1100 is in the radially expanded configuration.

For example, as depicted in FIGS. 23A, 24A, and 25, the tines 1102 can nest within cells of the anchoring frame 1100 when the anchoring frame 1100 is in a radially compressed state. This can be accomplished, for example, by forming the tines 1102 with an axial length that is less than or equal to the distance between adjacent strut junctions 1108 when the anchoring frame 1100 is in the radially compressed configuration. In some examples, the tines 1102 can be radially aligned (e.g., flush) with an outer surface 1110 of other struts when the anchoring frame 1100 is in the radially compressed configuration. In other examples, the tines 1102 can be radially offset (e.g., recessed) from the outer surface 1110 of other struts when the anchoring frame 1100 is in the radially compressed configuration.

As the anchoring frame 1100 radially expands from the radially compressed state, the cells of the anchoring frame 1100 axially foreshorten, as shown in FIGS. 23A-23C. This causes axially aligned strut junctions 1108 to move toward each other and results in the free end portions 1106 of the tines 1102 contacting adjacent strut junctions 1108. The free end portions 1106 and/or the strut junctions 1108 can be configured such that the free end portions 1106 of the tines deflect radially outwardly relative to the strut junctions 1108, as shown in FIGS. 23B and 24B. This can be accomplished, for example, by forming the free end portions 1106 of the tines and/or the strut junctions 1108 with ramped or tapered surfaces and/or other means for guiding the free end portions of the tines toward the outer surface 1110 of the anchoring frame 1100. FIGS. 23B and 24B depict a portion of the anchoring frame 1100 in a partial radially expanded state, in which the free end portions 1106 of the tines are beginning to axially slide over the strut junctions 1108 and to deflect radially outwardly relative to the strut junctions 1108. FIGS. 23C and 24C depict the portion of the anchoring frame 1100 further radially expanded compared to FIGS. 23B and 24B. In this configuration, the free end portions 1106 of the tines axially overlap with the strut junctions 1108 and deflect radially outwardly relative to the strut junctions 1108. The outwardly projecting free end portions 1106 of the tines 1102 can engage native tissue and help to prevent a prosthetic heart valve to which the anchoring frame 1100 is coupled from migrating relative to the native tissue.

Referring to FIG. 22, all of the tines 1102 of the anchoring frame 1100 are configured in the same general orientation, namely the fixed end portion 1104 of each tine 1102 is disposed relatively closer to a first end portion 1112 of the anchoring frame 1100 than its respective free end portion 1106. In other examples, all of the tines of an anchoring frame can be configured such that the fixed end portion of each tine is disposed relatively closer to a second end portion 1114 of the anchoring frame than its respective free end portion. In yet other examples, the anchoring frame can be configured such that the fixed end portion of one or more tines is disposed relatively closer to the first end portion of the anchoring frame 1100 than its respective free end portion and such that the fixed end portion of one or more other tines is disposed relatively closer to the second end portion of the anchoring frame than its respective free end portion. Various patterns and/or orientations of tines can be combined.

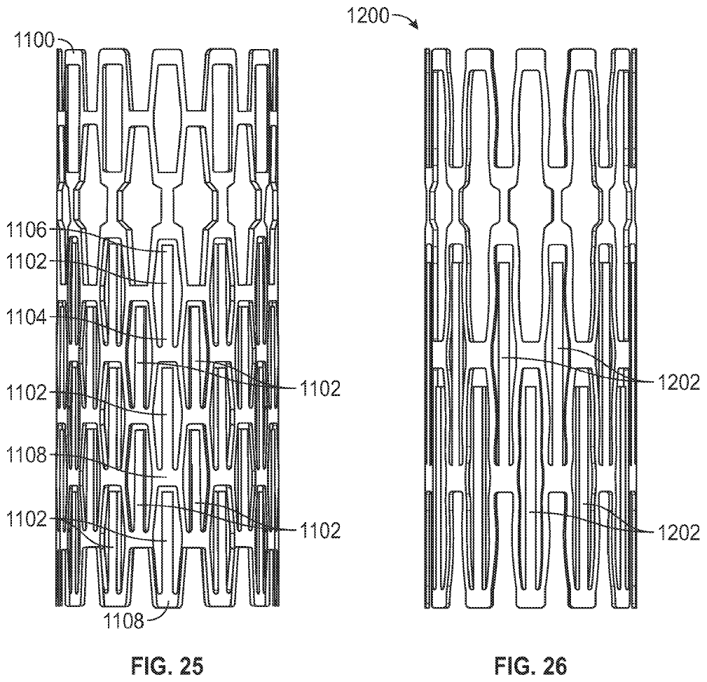

As depicted in FIGS. 22 and 25, the anchoring frame 1100 comprises five circumferentially extending rows of tines 1102, which are arranged in axially aligned columns of tines comprising either two tines or three tines. The columns alternate between two tines and three tines. In other examples, various other configurations can be used. For example, FIG. 26 depicts an anchoring frame 1200 comprising a plurality of tines 1202 that are arranged in two circumferentially extending rows. Various other row and/or column configurations can be used.

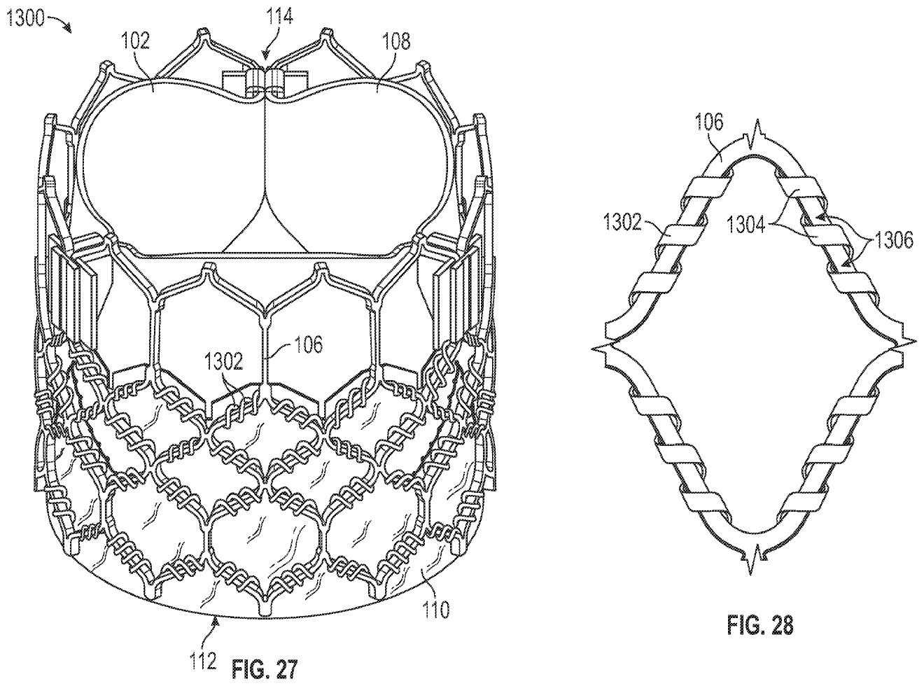

FIGS. 27-28 depict a prosthetic heart valve assembly 1300. The prosthetic heart valve assembly 1300 comprises the prosthetic heart valve 102 and one or more friction-increasing elements 1302 in lieu of the anchoring frame 104. The friction-increasing elements 1302 can be wrapped around the struts of the valve frame 106 and can engage native tissue adjacent the prosthetic heart valve assembly 1300 and help to secure the prosthetic heart valve assembly in place.

Referring to FIG. 28, in some examples, wraps 1304 of the friction-increasing elements 1302 are spaced from each other along the valve frame 106, which forms grooves 1306 therebetween. Thus, when the prosthetic heart valve assembly 1300 is radially expanded, the surrounding native tissue (e.g., native leaflets) can extend radially into the grooves 1306. The native tissue is thereby anchored within the grooves 1306 by the wraps 1304. In this manner, the friction-increasing elements 1302 can prevent movement (e.g., axial displacement) of the prosthetic heart valve assembly 1300 relative to the surrounding native tissue. Advantageously, the wraps 1304 and the grooves 1306 can, for example, provide sufficient retaining force to prosthetic heart valve assembly 1300, while also minimizing potential harm to the surrounding native tissue.

In some examples, the prosthetic heart valve assembly 1300 can comprise a single friction-increasing element wrapped around one or more strut sections of the valve frame 106 in a continuous manner. In other examples, a prosthetic heart valve assembly can comprise a plurality of friction-engaging elements, each wrapped around one or more sections of the valve frame 106.

In some examples, the friction-increasing elements 1302 can be disposed on (e.g., wrapped around) one or more portions of the valve frame 106 and not disposed on one or more other portions of the valve frame 106. For example, as depicted in the illustrated example, the friction-increasing elements 1302 can be wrapped around strut sections of the inflow end portion 112 of the valve frame 106, and not around the outflow end portion 114 of the valve frame 106. In other examples, the friction-increasing elements can be disposed on the entire axial length of the valve frame.

Various aspects of the friction-increasing elements 1302 can be altered to help ensure sufficient retention force of the prosthetic heart valve assembly 1300 against the surrounding tissue, once deployed. For example, various types of materials, rigidities, widths, thicknesses, as well as wrapping configurations, including the amount and density of wraps and the amount and location of cells or strut sections to be wrapped, can be chosen.

In some examples, the friction-increasing elements 1302 can be wires or sutures. The wires or sutures can be made of a metal wire or cable (e.g., MP35N, stainless steel, Nitinol, etc.) and/or polymeric fibers (e.g., PET, UHMWPE (e.g., Dyneema®), polyurethane (“PU”), nylon, etc.).

In particular examples, the friction-increasing elements 1302 can have a non-smooth surface pattern so as to increase friction between the friction-increasing elements 1302 and native tissue which the friction-increasing elements 1302 contact. For example, the friction-increasing elements 1302 can have a braided, twisted, and/or coiled surface pattern.

The friction-increasing elements 1302 can be wrapped around the struts in various directions (e.g., right-handed or left-handed). In some examples, all of the friction-increasing elements 1302 can be wrapped in a uniform direction (e.g., right-handed). In other examples, a first friction-increasing element 1302 or a first portion of the first friction-increasing element can be wrapped in a first direction (e.g., right-handed) and a second friction-increasing element 1302 or a second portion of the first friction-increasing element can be wrapped in a second direction (e.g., left-handed). In some examples, the first friction-increasing element and the second friction-increasing element can crisscross.

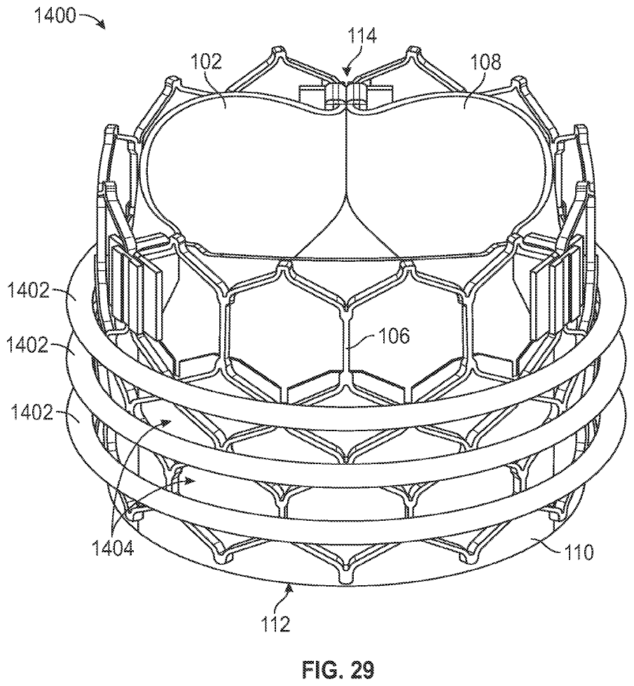

FIG. 29 depicts a prosthetic heart valve assembly 1400. The prosthetic heart valve assembly 1400 comprises the prosthetic heart valve 102 and one or more friction-increasing elements 1402 (e.g., three in the illustrated example) extending circumferentially around the prosthetic heart valve 102. The friction-increasing elements 1402 can also be referred to as “the friction rings 1402.”

The friction rings 1402 can be coupled to the prosthetic heart valve 102 by a frictional engagement. For example, the friction rings can have an inner diameter that is slightly less than the outer diameter of the valve frame, and the friction rings can be elastically deformed (e.g., stretched) to fit over the valve frame. The friction rings can be sufficiently elastically deformable to allow the valve frame to be expanded from the radially compressed state to the radially expanded state. Additionally or alternatively, the friction rings 1402 can be coupled to the prosthetic heart valve 102 (e.g., to the valve frame 106 and/or sealing member 110) via sutures, adhesive, and/or other means for coupling.

In the illustrated example, the prosthetic heart valve assembly 1400 comprises three friction rings 1402. In other examples, a prosthetic heart valve can comprise less or more than three friction rings. For example, a prosthetic heart valve assembly can comprise 1, 2, or 4-12 friction rings.

The friction rings 1402 are spaced apart from each other to form gaps 1404 therebetween. Various spacing between the friction rings can be used. For example, the gaps 1404 between each adjacent pair of friction rings 1402 can be uniform or at least substantially uniform. In other examples, the gaps 1404 can be non-uniform.

In examples comprising a plurality of friction rings 1402, the surrounding native tissue (e.g., native leaflets) can extend radially into the gaps 1404 when the prosthetic heart valve assembly 1400 is expanded and contacts the native tissue. The native tissue can be secured between the adjacent sidewalls of the friction rings 1402, which can prevent or reduce axial movement or displacement of the prosthetic heart valve assembly 1400 relative to native tissue.

The axial location of the friction rings 1402 relative to prosthetic heart valve 102 can vary. For example, the friction rings 1402 can be disposed either closer to the inflow end portion 112 or closer to the outflow end portion 114 of the prosthetic heart valve than depicted in the illustrated example.

In some examples, the friction rings can be disposed over the entire axial length of the prosthetic heart valve (i.e., spaced from the inflow end portion to the outflow end portion). In other examples, the friction rings 1402 can be disposed over only one or more portions of the prosthetic heart valve and not over one or more other portions of the prosthetic heart valve. For example, the friction rings 1402 can be wrapped around the inflow end portion 112 of the prosthetic heart valve 102 and not around the outflow end portion 114 of the prosthetic heart valve 102.

One or more aspects of the friction rings, including type of material and thickness/width, as well as the number, density, and location of friction rings, can be chosen to provide sufficient retention force of the prosthetic heart valve against the surrounding tissue, once deployed. One or more of these aspects can also be chosen to promote tissue ingrowth within the gaps over time, which can advantageously provide additional stability against relative valve displacement.

In some examples, the friction rings comprise a polymeric material such as silicone, PU, and/or another material configured to provide sufficient friction against the surrounding anatomy so as to prevent axial displacement of the prosthetic heart valve assembly 1400 when deployed against the native anatomy. According to some examples, the friction rings can be provided in the form of O-rings.

In some examples, one or more of the friction rings can be made of a different material and/or have different dimensions (e.g., thickness) than one or more other friction rings.

Since the various anchoring frames and the friction increasing elements disclosed herein do not involve structural modifications to the prosthetic heart valve and because these components can be coupled to a prosthetic heart valve relatively easily, the disclosed devices and methods advantageously enable utilization of existing lines of products (e.g., the Edwards Sapien 3® prosthetic heart valve) to form a prosthetic heart valve assembly comprising one or more of the disclosed friction-increasing elements and/or one of the anchoring frames. Thus, the devices and assemblies disclosed herein not only reduce migration of the prosthetic heart valve, they are also modular, cost-effective, and/or relatively simple to implement.

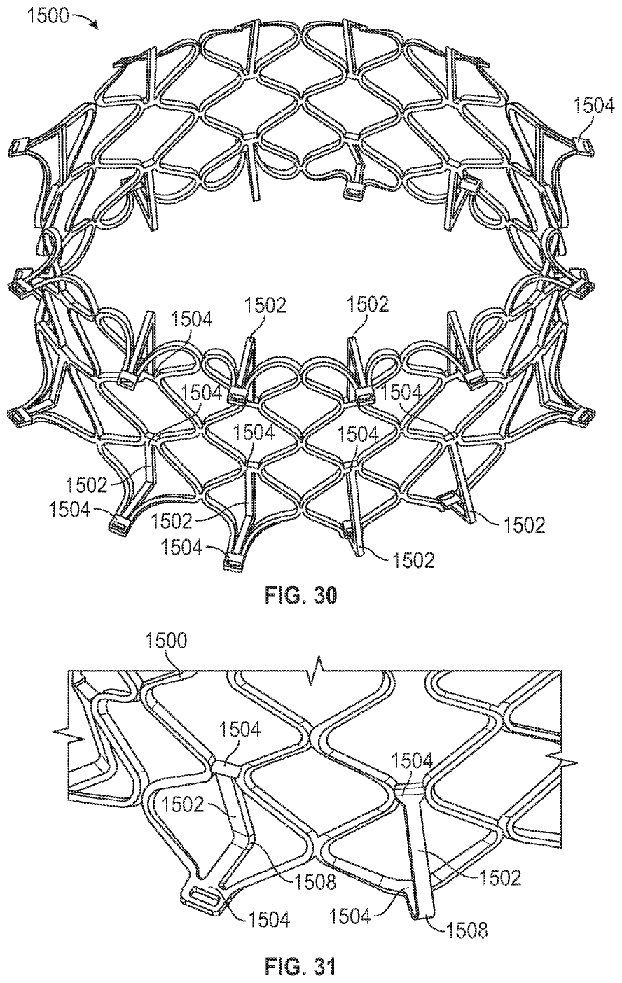

FIGS. 30-32 depict a frame 1500 for a prosthetic heart valve and/or prosthetic heart valve assembly. The frame 1500 can, for example, be used as a valve frame of the prosthetic heart valve 102 in lieu of the valve frame 106. As another example, the frame 1500 can be used as an anchoring frame of the prosthetic heart valve assembly 100 in lieu of the anchoring frame 104.

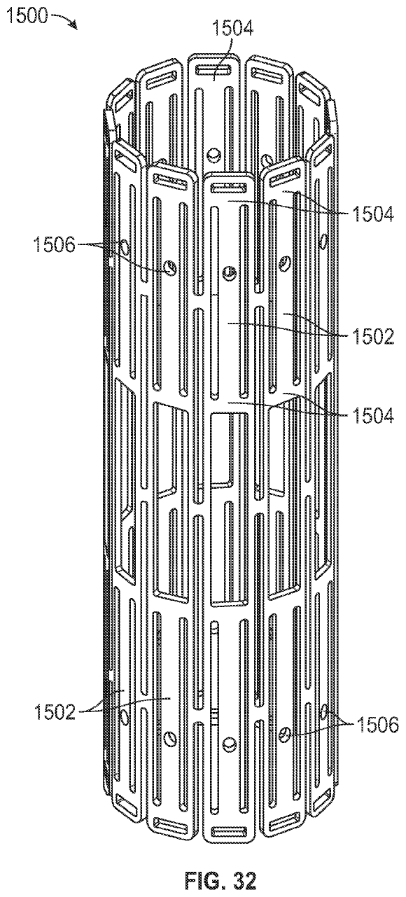

As a general overview, the frame 1500 is configured to have a cylindrical or at least substantially cylindrical profile when the frame 1500 is in a radially compressed configuration (e.g., FIG. 32). The frame 1500 is also configured to have a non-cylindrical profile when the frame 1500 is in a radially expanded configuration (e.g., FIG. 30). This can be accomplished, for example, by forming the frame 1500 with one or more plastically deformable portions and one or more elastically deformable portions. Accordingly, the frame 1500 can have a cylindrical geometry in a radially compressed configuration (e.g., FIG. 32) and a non-cylindrical geometry (or a less-cylindrical geometry) due to plastic deformation of the plastically deformable portions when the frame 1500 is in a radially expanded configuration (e.g., FIG. 30).

More specifically, the frame 1500 comprises a plurality of struts forming cells, and one or more of the cells have anchoring members 1502 attached at strut junctions 1504. The anchoring members 1502 are formed of plastically deformable material (e.g., MP35N™, stainless steel, etc.), while one or more other portions of the frame 1500 are formed from an elastically deformable material (e.g., Nitinol). The anchoring members 1502 are configured to plastically deform (e.g., buckle) during radial expansion of the frame 1500 due to the axially-compressive forces on the anchoring members 1502 applied by the strut junctions 1504 moving axially toward each other. The buckling causes the anchoring members 1502 to flare radially (either outwardly or inwardly) relative to a central longitudinal axis of the frame 1500, which results in the frame 1500 moving from a cylindrical configuration (e.g., FIG. 32) to a non-cylindrical configuration (e.g., FIG. 30). Stated another way, intermediate portions of the anchoring members are radially offset relative to end portions of the anchoring members when the frame is in the radially expanded configuration.

In the illustrated example, the anchoring members 1502 are straight (e.g., vertical) when the anchoring members are in the undeformed state (e.g., FIG. 32). In other examples, the anchoring members can comprise various other shapes. For example, in some instances, the anchoring members can comprise an “S” shape.

In some examples, the anchoring members 1502 can comprise one or more buckling guides configured to cause the anchoring members to plastically deform at a particular location. For example, as depicted in FIG. 32., the anchoring members 1502 comprise buckling guides in the form of apertures 1506. The anchoring members 1502 tend to buckle at the apertures 1506 when the frame 1500 is radially expanded because the anchoring members 1502 are relatively weak adjacent the apertures 1506 due to there being less material. It should be noted that for purposes of illustration, the apertures 1506 are not shown in FIGS. 30-31.

As depicted in FIGS. 30-31, some of the anchoring members 1502 are oriented such that apices 1508 of the anchoring members 1502 are disposed radially outwardly from their respective strut junctions 1504, and some others of the anchoring members 1502 are oriented such that the apices 1508 of the anchoring members 1502 are disposed radially inwardly from their respective strut junctions 1504. The number and/or pattern of radially inwardly facing anchoring members and/or radially outwardly facing anchoring members can vary. For example, in some instances, all of the anchoring members can extend radially outwardly. In other examples, all of the anchoring members can extend radially inwardly. In other examples, all of the anchoring members at an inflow end portion of the frame can extend radially outwardly, all of the anchoring members at an outflow end portion of the frame can extend radially inwardly. In yet other examples, the anchoring members can be configured in alternating in-out pattern (e.g., a one in-two out pattern, or vice versa).

In particular examples, the buckling guides can be configured to help direct the anchoring members in specific direction (e.g., radially inwardly or radially outwardly) as the anchoring members plastically deform. For example, in lieu of or in addition to the apertures 1506, the anchoring members 1502 can comprise one or more grooves, slots, notches, etc. formed in the radially inwardly facing surface and/or the radially outwardly facing surface of the anchoring members. In such examples, the anchoring members tend to buck away from the notch or groove. For example, an anchoring member with a groove formed in the radially inwardly facing surface of the anchoring member tends to buckle such that the apex of the anchoring member flares radially outwardly. As another example, an anchoring member with a groove formed in the radially outwardly facing surface of the anchoring member tends to buckle such that the apex of the anchoring member flares radially inwardly.

In some examples, the frame 1500 can be formed in the cylindrical delivery configuration depicted in FIG. 32. The frame 1500 can be crimped onto an inflatable balloon of a delivery apparatus. The frame 1500 can be moved from the delivery configuration to the non-cylindrical functional configuration by inflating the balloon, which plastically deforms the anchoring member 1502. The anchoring members 1502 can engage native tissue surrounding the frame at an implantation location. In this manner, the anchoring members 1502 can help to prevent or reduce migration of the frame 1500 (and thus a prosthetic heart valve) relative to the native tissue.

The frame 1500 serves only as an exemplary example. In other examples, a frame can be shape-set to any desired expanded form by designing the plastically deformable portions according to several parameters that influence its behavior, such as the type of material and the dimensions that influence the degree and direction of deformability, and/or by designing their attachments to the other components of the frame, including: the total number and distribution of the plastically deformable portions, attachment points along the frame, and/or orientation relative to the cells or other components of the frame.

The prosthetic heart valve assemblies disclosed herein can resist migration relative to the native tissue. Thus, the disclosed assemblies can be used, for example, in patients with relatively non-stenotic anatomy and/or at implantation locations that do not provide sufficient structure for a typical prosthetic heart valve (e.g., native mitral valve). Additionally, the anchoring frames and/or other tissue-engaging elements disclosed herein can, for example, be attached (e.g., retrofitted) to a prosthetic heart valve. Therefore, the disclosed devices and methods improve stability and reduce migration of prosthetic heart valves, while also being relatively simple and cost-effective to implement.

It should be noted that, although the devices and methods described herein are primarily directed to prosthetic heart valve assemblies that are configured for implantation within a native heart valve (e.g., an aortic, a mitral valve, a tricuspid valve, and/or a pulmonary valve), the disclosed devices and methods can be configured for implantation at various other locations, including within a blood vessel. For example, in certain examples, an anchoring frame (e.g., the anchoring frame 104) can be configured for implantation within a vena cava.

In some examples, a docking assembly is provided. The docking assembly can comprise a prosthetic docking device, one or more of the anchoring frames disclosed herein, and/or one or more of the tissue-engaging elements disclosed herein, which can be coupled to the prosthetic docking device (e.g., with sutures, fasteners, adhesive, and/or other means for coupling).

In other examples, a graft assembly is provided. The graft assembly can comprise a graft (e.g., an aortic graft) one or more of the anchoring frames disclosed herein, and/or one or more of the tissue-engaging elements disclosed herein, which can be coupled to the graft (e.g., with sutures, fasteners, adhesive, and/or other means for coupling).

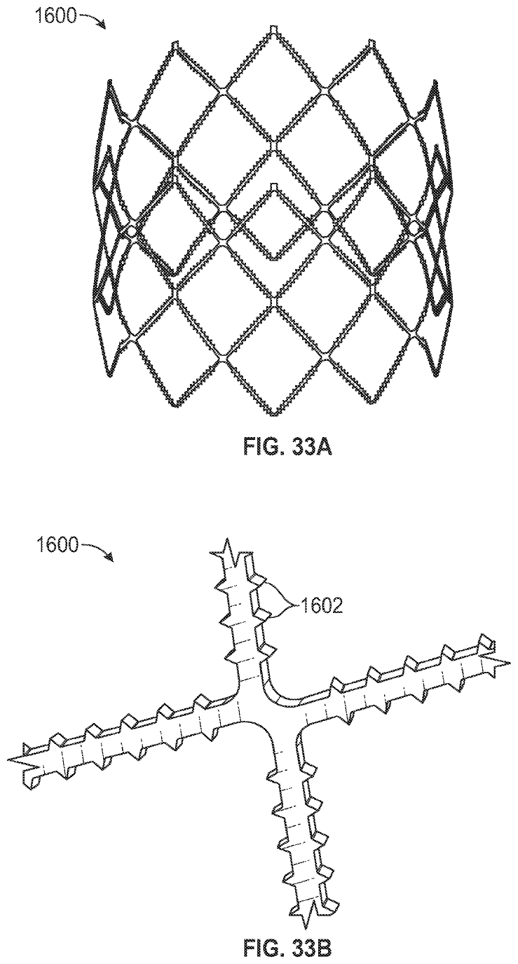

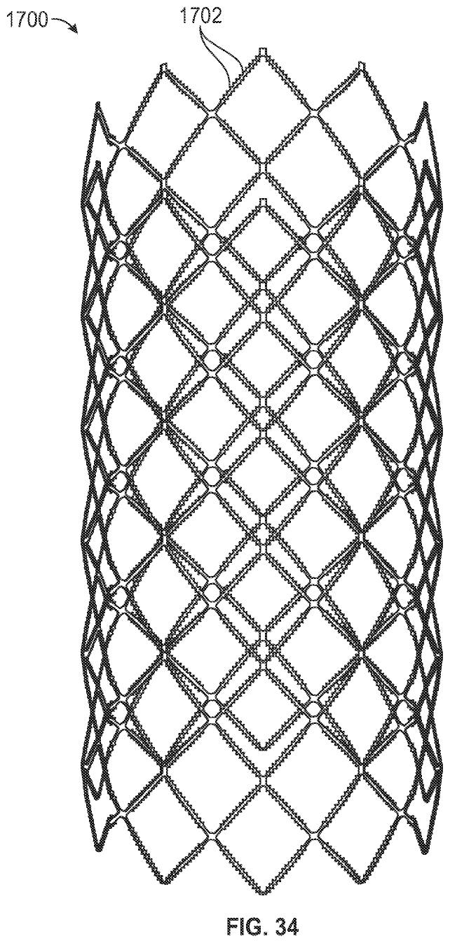

FIGS. 33A-33B depict a relatively short anchoring frame 1600, and FIG. 34 depicts a relatively long anchoring frame 1700. The anchoring frame 1600 and the anchoring frame 1700 respectively comprise projections 1602, 1702 extending from the struts of the anchoring frame, which can be configured similar to the projections 124 of the anchoring frame 104.

In some examples, prosthetic devices, such as a docking device, can include a relatively short external frame configured for engagement against the native tissue and an internal frame configured to retain a prosthetic valve that can be deployed therein. In some such examples, the relatively short anchoring frame 1600 can be coupled (e.g., sutured) to the outer frame of the docking device.

Some prosthetic devices comprise frames with two portions (e.g., a proximal portion and a distal portion), each portion including a relatively short sub-frame portion, attached therebetween by longer strut sections. In some such examples, two of the relatively short anchoring frames 1600 can be used (e.g., each anchoring frame attached to a respective sub-frame portion.

Some prosthetic devices, such as grafts and/or stents, include relatively long frames. In such instances, the relatively long anchoring frame 1700 can be coupled to the graft or stent.

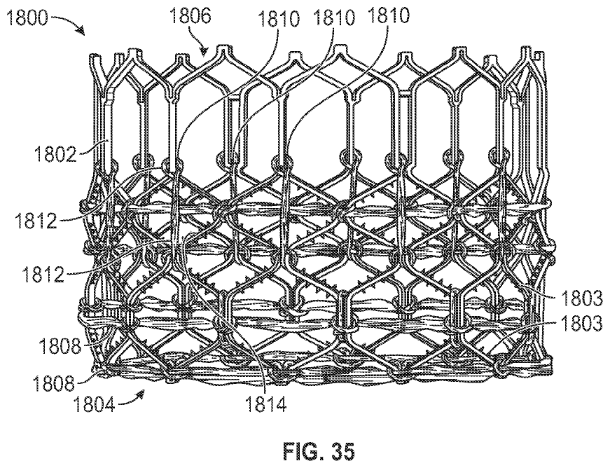

FIG. 35 illustrates a prosthetic valve 1800, according to one example. The prosthetic valve 1800 comprises a frame 1802, an inflow end portion 1804, and an outflow end portion 1806. The prosthetic heart valve 1800 can also include a valvular structure (e.g., the valvular structure 108 described above) and an inner sealing skirt (e.g., the inner skirt 110 described above), though these components are omitted for purposes of illustration. The frame 1802 can be a plastically expandable frame formed, for example, from stainless steel or a cobalt-chromium alloy and can be radially expanded using a balloon or other expansion mechanism. Thus, the prosthetic valve 1800 can be referred to a balloon-expandable valve. Further details regarding the prosthetic valve are disclosed in U.S. Pat. No. 9,393,110 and U.S. Publication No. 2018/0028310. In other examples, the prosthetic valve 1800 can be a self-expandable valve having a frame made of a shape-memory material, such as Nitinol.

As shown in FIG. 35, the frame 1802 can, in some instances, comprise a plurality of anchoring members 1803 (or projections) that extend from selected struts of the frame 1802. In other examples, the anchoring members 1803 can be disposed on an anchoring frame which is coupled to the frame 1802 of the prosthetic heart valve 1800. The anchoring members 1803 can be configured to secure the prosthetic valve 1800 to native tissue at a selected implantation site and/or help promote tissue ingrowth between the native tissue and the prosthetic valve 1800. The anchoring members 1803 can extend in various directions from the struts of the frame 1802. For example, in some instances, one or more of the anchoring members 1803 can extend from the struts at an angle relative to a central longitudinal axis extending from the inflow end to the outflow end of the prosthetic heart valve assembly. In some instances, the anchoring members 1803 can be perpendicular or at least substantially perpendicular (e.g., forming an angle of 80-100 degrees) to the struts from which they extend. In other examples, the anchoring members 1803 can extend from their respective struts at various other angles (e.g., between 1-79 degrees). For example, in some instances, the anchoring members can extend from their respective struts at an angle of about 45 degrees such that anchoring members are parallel or at least substantially parallel to a central longitudinal axis extending from the inflow end to the outflow end of the prosthetic heart valve assembly.

The frame 1802 (or a portion of the frame) can be configured as an anchoring frame. As such, the frame 1802 can, in some instances, be coupled to another frame (e.g., the frame of a prosthetic heart valve, a stent, or a graft).

The anchoring members 1803 can comprise various shapes and lengths such that the projections provide sufficient retention force for the prosthetic heart valve assembly, while reducing potential harm to the surrounding tissue. For example, in the illustrated example, the anchoring members 1803 comprise tines or spikes. In other example, the anchoring members 1803 can comprise ball-shaped bulges and/or a rectangular shape. Additionally or alternatively, one or more of the anchoring members 1803 can comprise a curved shape, a hook shape, a cross shape, a T-shape, and/or a barbed shape. Various combinations of shapes and/or sizes of anchoring members 1803 can be used.

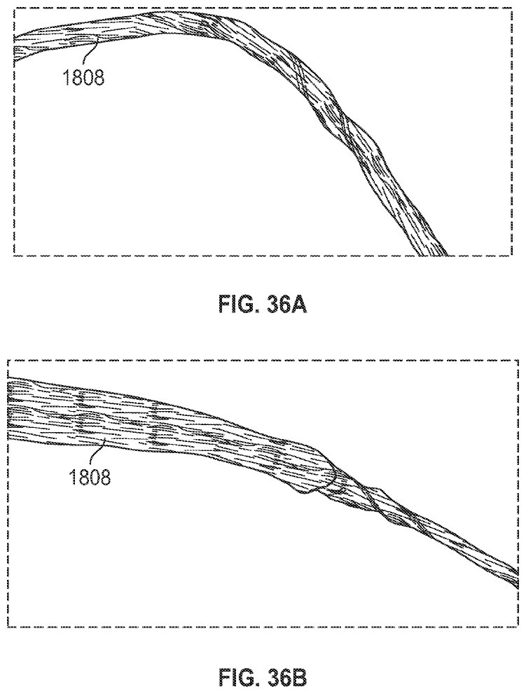

In lieu of or in addition to a sealing member or outer skirt disposed on the outside of the frame (and/or an anchoring frame), the prosthetic valve 1800 can comprise one or more expandable yarns or sutures 1808. Referring to FIGS. 36A and 36B, the suture 1808 can be resiliently stretchable and can be placed in a tensioned and axially elongated state (FIG. 36A) when tensioned is applied and a relaxed, non-tensioned state (FIG. 36B) when tension is removed. When placed in tension, the suture 1808 is axially elongated and has a reduced diameter (FIG. 36A), but when tension is released (e.g., when the suture is in a relaxed state) the sutures can increase in diameter and become fuzzy and puffy (FIG. 36B), increasing their ability to absorb fluid (e.g., blood). The sutures 1808 can function as a sealing member for the prosthetic valve by sealing against the tissue of the native valve annulus and helping to reduce paravalvular leakage. In some example, the sutures 1808 can comprise a plurality of texturized filaments, which can, for example, be twisted or braided together. The texturized filaments can be texturized, for example, via pin texturizing. For example, in some instances, the expandable sutures can comprise draw texturized yarn (DTY) including a plurality of filaments that have been twisted together (e.g., 3,000-4,000 times per meter) and heat treated to create fine crimps in the filaments. In other instances, the filaments can be texturized via gear texturizing and/or air texturizing.

As shown in FIG. 35, the prosthetic valve 1800 comprises a plurality of sutures 1808 circumscribing the outer surface of frame 1802 and spaced apart from one another along a longitudinal axis of the prosthetic valve 1800. The prosthetic valve 1800 can further comprise vertically extending sutures 1810. The vertically extending sutures 1810 can extend between opposing junctions 1812 of a respective cell 1814 of the frame 1802. In the illustrated example, a sealing member on the outer surface of the frame is omitted. The sutures 1808 cover a reduced surface area of the outer surface of the frame 1802 relative to a typical outer skirt, which advantageously allows the anchoring members 1803 to engage the native tissue.

As mentioned, in some instances, the sutures 1808 can comprise a plurality of texturized filaments that are combined (e.g., twisted and/or braided) together to form the expandable suture. The sutures 1808 can comprise any number of filaments, for example, between 2 and 20 filaments. In particular examples, the sutures comprise 12 filaments. When tensioned, the sutures 1808 can have a diameter equivalent to that of a suture typically used for securing soft components of a prosthetic valve to each other or to a frame of the valve (e.g., a 2-0, 3-0, or 4-0 suture), for example, between about 0.15 mm to about 0.3 mm. In some particular examples, the expandable sutures 1808 can comprise polyester.

In some examples, the sutures 1808 can each comprise 8 filaments or “ends” of pin textured polyethylene terephthalate (PET). The linear mass density of the filaments can be 1/40 Den/27 Fil. The filaments can be braided together at a braid density of 10 picks per inch (PPI) with alternating carrier tension (e.g., during braiding some of the filaments can be held in tension while others are held loosely, or some filaments can be held at a first tension while others are held at a second tension, etc.) to form the suture 1808. The suture(s) 1808 can be heat set at, for example, 320° F. while wrapped around a spool. In other examples, the suture(s) can be heat set at, for example, 320° F. in individual units.

In other examples, the sutures 1808 can each comprise 12 filaments or “ends” of pin textured PET having a linear mass density of 1/20Den/27Fil. In some instances, the filaments can be braided together at a braid density of 10 PPI with alternating carrier tension to form the suture 1808. In other instances, the filaments can be braided with alternating carrier tension and variable pick density (e.g., variable PPI) to form the suture 1808. The suture(s) 1808 can be heat set, for example, at 320° F. while wrapped around a spool. In other examples, the suture(s) can be heat set at 320° F. in individual units.

In still other examples, the sutures 1808 can each comprise 12 filaments or “ends” of pin textured PET having a linear mass density of 1/20Den/27Fil. In some instances, the filaments can be braided together with alternating carrier tension and variable pick density (e.g., variable PPI) to form the suture. In other instances, the filaments can be braided together at a braid density of 10 PPI with alternating carrier tension to form the suture 1808. The suture(s) 1808 can be heat set at, for example, 320° F. while wrapped around a spool. In other examples, the suture(s) can be heat set at 320° F. in individual units.

In some examples, the sutures 1808 are assembled on the outer surface of the frame in the non-tensioned and expanded state (FIG. 36B). Once the prosthetic valve 1800 is implanted at a selected implantation site, the non-tensioned or relaxed sutures 1808 (FIG. 36B) can promote tissue ingrowth and reduce PVL. In some examples, the relaxed sutures 1808 can absorb and swell with blood. When in the relaxed configuration, as shown in FIG. 36B, yarns or filaments of the suture 1808 loosen from one another and fan radially outward from a longitudinal axis of the suture 1808 to create the fuzzy or puffy texture. The relaxed sutures 1808 can serve as a sealing member configured to prevent or mitigate PVL.

The sutures 1808 can be assembled on the frame such that selected portions of the suture 1808 are retained in the tensioned configuration and other portions are retained in the relaxed configuration. The tensioned and relaxed sections of the sutures 1808 can be retained in their tensioned or relaxed state by “locking” the ends of each tensioned or relaxed section to the frame for example, by knotting the suture 1808 to the frame, wrapping the suture 1808 around a junction or strut of the frame, and/or by using an additional suture to tie-off a portion of the suture 1808 in order to maintain certain portions of the suture 1808 in the relaxed or tensioned configurations. For example, in some instances, sections of the suture 1808 on the outer surface of the frame can be retained in a relaxed state to promote sealing, while sections of the suture 1808 on inside of the frame can be retained in a tensioned state to reduce crimp profile and/or for tightly securing other components (e.g., an inner skirt) to the frame. When forming a tensioned section, the suture 1808 can be locked (e.g., knotted or wrapped around a strut or junction) to the frame at a first location, pulled taught and then locked to the frame at a second location. When forming a relaxed section, the suture 1808 can be locked to the frame at first and second locations with the section of the suture between the first and second locations being in a non-tensioned relaxed state. A single suture 1808 can be used to form one or more tensioned sections and one or more relaxed sections at various locations on the prosthetic valve.

Though the illustrated example of FIG. 35 shows four sutures 1808 configured as rings, it should be noted that in other examples more or less than four sutures can be used. For example, in some instances, the prosthetic valve can comprise a single expandable suture circumscribing an inflow end portion 1804 of the prosthetic valve 1800.

Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve assembly comprises a prosthetic heart valve and an anchoring frame. The prosthetic heart valve comprising a valve frame and a valvular structure. The valve frame comprising a plurality of struts and being expandable from a radially compressed delivery configuration to a radially expanded configuration. The valvular structure comprising a plurality of leaflets and being disposed radially within and is coupled to the valve frame. The valvular structure is configured to allow blood flow through the prosthetic heart valve assembly in a first direction and to restrict blood flow through the prosthetic heart valve assembly in a second direction. An anchoring frame comprising a plurality of struts and being disposed radially outwardly from and is coupled to the valve frame. The struts of the anchoring frame comprise one or more tissue-engaging elements configured for contacting native tissue to help secure the prosthetic heart valve assembly at an implantation location.

Example 2. The prosthetic heart valve assembly of any example herein, particularly example 1, wherein the tissue-engaging elements include one or more projections extending from the struts of the anchoring frame.

Example 3. The prosthetic heart valve assembly of any example herein, particularly example 2, wherein the projections extend from the struts at an angle relative to a central longitudinal axis of the prosthetic heart valve assembly.

Example 4. The prosthetic heart valve assembly of any example herein, particularly example 2, wherein the projections extend from the struts such that the projections are at least substantially parallel to a central longitudinal axis of the prosthetic heart valve assembly.

Example 5. The prosthetic heart valve assembly of any example herein, particularly example 1 or example 2, wherein the tissue-engaging elements include a plurality of grooves formed in the struts of the anchoring frame.

Example 6. The prosthetic heart valve assembly of any example herein, particularly example 5, wherein the grooves are spaced apart relative to each other so as to form ridges between the grooves.

Example 7. The prosthetic heart valve assembly of any example herein, particularly any one of examples 1-6, wherein the tissue-engaging elements include a plurality of tines extending from the struts of the anchoring frame, and wherein the tines are arranged in pairs of tines that are axially aligned with each other.

Example 8. The prosthetic heart valve assembly of any example herein, particularly example 7, wherein each pair of tines includes a first tine and a second tine, wherein the first tine is disposed toward the inflow end portion of the prosthetic heart valve assembly relative to the second tine, wherein the first tine and the second tine are spaced apart by a first distance when the anchoring frame is in a radially compressed configuration, wherein the first tine and the second tine are spaced apart by a second distance when the anchoring frame is in a radially expanded configuration, and wherein the second distance is less than the first distance.

Example 9. The prosthetic heart valve assembly of any example herein, particularly example 8, wherein the anchoring frame comprises a plurality of cells, wherein the cells have a first axial length when the anchoring frame is in the radially compressed configuration and a second axial length when the anchoring frame is in the radially expanded configuration, the second axial length being less than the first axial length, wherein each pair of tines is disposed within a respective cell, and wherein a combined axial length of the first tine and the second tine of each pair of tines is less than the second axial length of the respective cell in which the pair of tines is disposed.

Example 10. The prosthetic heart valve assembly of any example herein, particularly any one of examples 1-6, wherein the tissue-engaging elements include a plurality of tines, wherein the tines are configured to be radially aligned with the struts of the anchoring frame when the anchoring frame is in a radially compressed configuration, and wherein the tines are configured to extend radially outwardly from the struts of the anchoring frame when the anchoring frame is in a radially expanded configuration.

Example 11. The prosthetic heart valve assembly of any example herein, particularly example 10, wherein the anchoring frame comprises a plurality of cells, wherein the cells have a first axial length when the anchoring frame is in the radially compressed configuration and a second axial length when the anchoring frame is in the radially expanded configuration, the second axial length being less than the first axial length, and wherein the tines have an axial length that is less than the first axial length of the cells and greater than the second axial length of the cells.

Example 12. The prosthetic heart valve assembly of any example herein, particularly example 11, wherein the tines axially overlap with the struts of the frame that define the cells when the anchoring frame is in the radially expanded configuration.

Example 13. The prosthetic heart valve assembly of any example herein, particularly any one of examples 10-12, wherein the struts of the anchoring frame comprise one or more ramped surfaces configured to direct the tines radially outwardly relative to the struts as the anchoring frame moves from the radially compressed configuration to the radially expanded configuration.

Example 14. The prosthetic heart valve assembly of any example herein, particularly any one of examples 10-13, wherein the tines comprise ramped surfaces configured to direct the tines radially outwardly relative to the struts as the anchoring frame moves from the radially compressed configuration to the radially expanded configuration.

Example 15. The prosthetic heart valve assembly of any example herein, particularly any one of examples 10-14, wherein each of the tines comprises a fixed end portion and a free end portion.

Example 16. The prosthetic heart valve assembly of any example herein, particularly example 15, wherein one or more of the tines is oriented such that the free end portion is disposed toward an outflow end portion of the anchoring frame relative to the fixed end portion.

Example 17. The prosthetic heart valve assembly of any example herein, particularly example claim 15 or example 16, wherein one or more of the tines is oriented such that the free end portion is disposed toward the inflow end portion of the anchoring frame relative to the fixed end portion.

Example 18. The prosthetic heart valve assembly of any example herein, particularly any one of examples 10-17, wherein each tine is disposed completely within a respective cell of the anchoring frame when the anchoring frame is in the radially compressed configuration, and wherein each tine extends partially from the respective cell of the anchoring frame when the anchoring frame is in the radially expanded configuration.

Example 19. The prosthetic heart valve assembly of any example herein, particularly any one of examples 1-18, further comprising a sealing member coupled to the valve frame or the anchoring frame.

Example 20. The prosthetic heart valve assembly of any example herein, particularly example 19, wherein the sealing member comprises an inner skirt and an outer skirt, wherein the inner skirt is disposed radially inwardly from the valve frame, and wherein the outer skirt is disposed radially outwardly from the anchoring frame.

Example 21. The prosthetic heart valve assembly of any example herein, particularly any one of examples 1-20, wherein the anchoring frame is removably coupled to the valve frame.

Example 22. The prosthetic heart valve assembly of any example herein, particularly example 21, wherein the anchoring frame is removably coupled to the valve frame with sutures.

Example 23. The prosthetic heart valve assembly of any example herein, particularly any one of examples 1-22, further comprising one or more friction-increasing elements coupled to the anchoring frame.

Example 24. The prosthetic heart valve assembly of any example herein, particularly example 23, wherein the friction-increasing elements are wrapped around the struts of the anchoring frame and/or the struts of the valve frame.

Example 25. The prosthetic heart valve assembly of any example herein, particularly example 24, wherein the friction-increasing elements include metal wires.

Example 26. The prosthetic heart valve assembly of any example herein, particularly any one of examples 23-25, wherein the friction-increasing elements include one or more rings circumscribing the anchoring frame.

Example 27. The prosthetic heart valve assembly of any example herein, particularly example 26, wherein the friction-increasing elements include a plurality of rings circumscribing the anchoring frame, wherein the rings are axially spaced apart relative to each other.

Example 28. The prosthetic heart valve assembly of any example herein, particularly any one of examples 1-27, wherein the valve frame comprises one or more cells, wherein the anchoring frame comprises one or more cells, and wherein the one or more cells of the anchoring frame are circumferentially and axially aligned with the one or more cells of the valve frame.

Example 29. The prosthetic heart valve assembly of any example herein, particularly any one of examples 1-27, wherein the valve frame comprises one or more cells, wherein the anchoring frame comprises one or more cells, and wherein the one or more cells of the anchoring frame are circumferentially or axially misaligned with the one or more cells of the valve frame.

Example 30. The prosthetic heart valve assembly of any example herein, particularly any one of examples 1-27, wherein the valve frame comprises one or more cells, wherein the anchoring frame comprises one or more cells, and wherein the one or more cells of the anchoring frame are smaller than the one or more cells of the valve frame.

Example 31. The prosthetic heart valve assembly of any example herein, particularly any one of examples 1-27, wherein the valve frame comprises one or more cells, wherein the anchoring frame comprises one or more cells, and wherein the one or more cells of the anchoring frame are larger than the one or more cells of the valve frame.

Example 32. The prosthetic heart valve assembly of any example herein, particularly any one of examples 1-31, wherein the valve frame comprises a first axial length, wherein the anchoring frame comprises a second axial length, and wherein the second axial length of the anchoring frame is less than the first axial length of the valve frame.

Example 33. The prosthetic heart valve assembly of any example herein, particularly example 32, wherein the anchoring frame is disposed on an inflow end portion of the valve frame and is axially spaced from an outflow end portion of the valve frame.

Example 34. The prosthetic heart valve assembly of any example herein, particularly example 32, wherein the anchoring frame is disposed on an outflow end portion of the valve frame and is axially spaced from an inflow end portion of the valve frame.

Example 35. The prosthetic heart valve assembly of any example herein, particularly any one of examples 1-31, wherein the valve frame comprises a first axial length, wherein the anchoring frame comprises a second axial length, and wherein the first axial length of the valve frame and the second axial length of the anchoring frame are at least substantially equal.

Example 36. The prosthetic heart valve assembly of any example herein, particularly any one of examples 1-35, wherein the valve frame comprises a first thickness, wherein the anchoring frame comprises a second thickness, and wherein the second thickness is less than the first thickness.

Example 37. The prosthetic heart valve assembly of any example herein, particularly any one of examples 1-35, wherein the valve frame comprises a first thickness, wherein the anchoring frame comprises a second thickness, and wherein a ratio of the second thickness to the first thickness is within a range of 0.3-0.9.

Example 38. An anchoring frame for a prosthetic heart valve assembly, the anchoring frame comprises a plurality of interconnected struts and a plurality of projections. The plurality of interconnected struts is configured to be moved from a radially compressed configuration to a radially expanded configuration and to be coupled to a prosthetic heart valve. The plurality of projections extends from the struts and is configured to engage native tissue at an implantation location.

Example 39. The anchoring frame of any example herein, particularly example 38, wherein one or more of the projections comprises a spike.

Example 40. The anchoring frame of any example herein, particularly example 38 or example 39, wherein one or more of the projections comprises a ball-shaped bulge.

Example 41. The anchoring frame of any example herein, particularly any one of examples 38-40, wherein one or more of the projections comprises a rectangular shape.

Example 42. The anchoring frame of any example herein, particularly any one of examples 38-41, wherein one or more of the projections comprises a curved shape.

Example 43. The anchoring frame of any example herein, particularly any one of examples 38-42, wherein one or more of the projections comprises a hook shape.

Example 44. The anchoring frame of any example herein, particularly any one of examples 38-43, wherein one or more of the projections comprises a barbed shape.

Example 45. The anchoring frame of any example herein, particularly any one of examples 38-44, wherein one or more of the projections comprises a cross shape.

Example 46. The anchoring frame of any example herein, particularly any one of examples 38-45, wherein one or more of the projections comprises a T-shape.

Example 47. The anchoring frame of any example herein, particularly any one of examples 38-46, wherein the struts comprise openings configured to receive sutures or fasteners that can be used to couple the anchoring frame to the prosthetic heart valve.

Example 48. The anchoring frame of any example herein, particularly any one of examples 38-47, wherein the struts form closed cells.

Example 49. The anchoring frame of any example herein, particularly any one of examples 38-48, wherein the struts form one or more circumferentially extending rows of closed cells.

Example 50. The anchoring frame of any example herein, particularly any one of examples 38-49, wherein the struts form exactly three circumferentially extending rows of closed cells.

Example 51. An anchoring frame for a prosthetic heart valve assembly, the anchoring frame comprises a plurality of interconnected struts comprising a plurality of grooves formed in radially outwardly facing surfaces of the struts. The struts are configured to be coupled to a prosthetic heart valve. The anchoring frame can be expanded from a radially compressed configuration to a radially expanded configuration. The grooves are configured to receive native tissue therein when the anchoring frame is radially expanded at an implantation location.

Example 52. The anchoring frame of any example herein, particularly example 51, wherein the grooves are spaced apart from each other such that the struts comprise ridges between the grooves.

Example 53. An anchoring frame for a prosthetic heart valve assembly, the anchoring frame comprises a plurality of interconnected struts and a plurality of tines extending from the struts. The plurality of interconnected struts is configured to be moved from a radially compressed configuration to a radially expanded configuration and to be coupled to a prosthetic heart valve. The tines are arranged in pairs including a first tine and a second tine. The first and second tine are axially aligned with each other. The first tine and the second tine are axially spaced apart relative to each other by a first distance when the struts are in the radially compressed configuration. The first tine and the second tine are axially spaced apart relative to each other by a second distance when the struts are in the radially expanded configuration, and the first distance is greater than the second distance.

Example 54. The anchoring frame of any example herein, particularly example 53, wherein the first tine and the second tine are configured such that native tissue can extend radially between the first tine and the second tine when the struts are in the radially compressed configuration, and wherein the first tine and the second tine are configured to capture the native tissue between the first tine and the second tine when the struts are in the radially expanded configuration.

Example 55. The anchoring frame of any example herein, particularly example 53 or example 54, wherein one or more of the tines extends toward an inflow end of the anchoring frame, and wherein one or more of the tines extends toward an outflow end of the anchoring frame.

Example 56. The anchoring frame of any example herein, particularly any one of examples 53-55, wherein the first tine and/or the second tine is/are parallel to a central longitudinal axis of the anchoring frame.

Example 57. The anchoring frame of any example herein, particularly any one of examples 53-55, wherein the first tine and/or the second tine extend at an angle within a range of 6-90 degrees relative to a central longitudinal axis of the anchoring frame.

Example 58. An anchoring frame for a prosthetic heart valve assembly, the anchoring frame comprises a plurality of interconnected struts and a plurality of tines. The plurality of interconnected struts is configured to be moved from a radially compressed configuration to a radially expanded configuration and to be coupled to a prosthetic heart valve. Each tine comprises a fixed end portion and a free end portion. The fixed end portions are coupled to one or more of the struts, and the free end portions are movable relative to the struts. When the struts are in the radially compressed configuration, the free end portions of the tines are in a first radial position. When the struts are in the radially expanded configuration, the free end portions of the tines are in a second radial position, and the second radial position is farther from a central longitudinal axis of the anchoring frame than the first radial position.

Example 59. The anchoring frame of any example herein, particularly example 58, wherein the free end portions of the tines are radially aligned or radially recessed relative to the struts when the prosthetic heart valve is in the radially compressed configuration.

Example 60. The anchoring frame of any example herein, particularly example 58 or example 59, wherein the free end portions of the tines nest within cells formed by the struts when the prosthetic heart valve is in the radially compressed configuration, and wherein the free end portions of the tines protrude from the cells formed by the struts when the prosthetic heart valve is in the radially expanded configuration.

Example 61. The anchoring frame of any example herein, particularly any one of examples 58-60, wherein the free end portions of the tines contact the struts when the free end portions move from the first radial position to the second radial position.

Example 62. The anchoring frame of any example herein, particularly example 61, wherein the free end portions of the tines comprise ramped surfaces configured to guide the free end portions of the tines radially outwardly relative to the struts when the free end portions of the tines contact the struts.

Example 63. The anchoring frame of any example herein, particularly example 61 or example 62, wherein the struts comprise ramped surfaces configured to guide the free end portions of the tines radially outwardly relative to the struts when the free end portions of the tines contact the struts.

Example 64. The anchoring frame of any example herein, particularly example 63, wherein the ramped surfaces are disposed at junctions of the struts.

Example 65. The anchoring frame of any example herein, particularly any one of examples 58-64, wherein the tines are arranged in one or more circumferentially-extending rows.

Example 66. The anchoring frame of any example herein, particularly any one of examples 58-65, wherein the tines are arranged in a plurality of circumferentially-extending rows.

Example 67. The anchoring frame of any example herein, particularly example 66, wherein the tines of a first circumferentially-extending row axially overlap with the tines of a second circumferentially-extending row.

Example 68. The anchoring frame of any example herein, particularly any one of examples 65-67, wherein the tines are arranged in 2-5 circumferentially-extending rows.

Example 69. A prosthetic heart valve comprises a valve frame, a valvular structure, and one or more friction-increasing elements. The valve frame comprises a plurality of struts and is expandable from a radially compressed delivery configuration to a radially expanded configuration. The valvular structure comprises a plurality of leaflets. The valvular structure is disposed radially within and is coupled to the valve frame. The valvular structure is configured to allow blood flow through the prosthetic heart valve assembly in a first direction and to restrict blood flow through the prosthetic heart valve assembly in a second direction. The one or more friction-increasing elements are wrapped around the struts of the frame. The friction-increasing elements are configured to engage native tissue and help to prevent the prosthetic heart valve from migrating relative to the native tissue.

Example 70. The prosthetic heart valve of any example herein, particularly example 69, wherein the friction-increasing elements comprise metal wire or metal cable.

Example 71. The prosthetic heart valve of any example herein, particularly example 69 or example 70, wherein the friction-increasing elements comprise polymeric fibers.

Example 72. The prosthetic heart valve of any example herein, particularly any one of examples 69-71, wherein the friction-increasing elements have a non-smooth surface pattern.

Example 73. The prosthetic heart valve of any example herein, particularly example 72, the non-smooth surface pattern includes one or more of a braided, twisted, or coiled surface pattern.

Example 74. A prosthetic heart valve comprises a valve frame, a valvular structure, and one or more friction-increasing elements. The valve frame comprises a plurality of struts. The valve frame is expandable from a radially compressed delivery configuration to a radially expanded configuration. The valvular structure comprises a plurality of leaflets. The valvular structure is disposed radially within and is coupled to the valve frame. The valvular structure is configured to allow blood flow through the prosthetic heart valve assembly in a first direction and to restrict blood flow through the prosthetic heart valve assembly in a second direction. One or more friction-increasing elements circumscribe the valve frame. The friction-increasing elements are configured to engage native tissue and help to prevent the prosthetic heart valve from migrating relative to the native tissue.

Example 75. The prosthetic heart valve of any example herein, particularly example 74, comprising a plurality of friction-increasing elements circumscribing the valve frame, wherein the friction-increasing elements are spaced apart from each other.

Example 76. The prosthetic heart valve of any example herein, particularly example 74 or example 75, wherein the friction-increasing elements comprise a polymeric material.

Example 77. A prosthetic heart valve comprises a valve frame and a valvular structure. The valve frame comprises a plurality of struts and a plurality of anchoring members. The valve frame is expandable from a radially compressed delivery configuration to a radially expanded configuration. The valve frame comprises a cylindrical shape in the radially compressed configuration and a non-cylindrical shape in the radially expanded configuration. The struts elastically deform when the valve frame moves from the radially compressed configuration to the radially expanded configuration. The anchoring members plastically deform when the valve frame moves from the radially compressed configuration to the radially expanded configuration. The valvular structure comprises a plurality of leaflets. The valvular structure is disposed radially within and is coupled to the valve frame. The valvular structure is configured to allow blood flow through the prosthetic heart valve assembly in a first direction and to restrict blood flow through the prosthetic heart valve assembly in a second direction.

Example 78. The prosthetic heart valve of any example herein, particularly example 77, wherein the anchoring members comprise first end portions, second end portions, and intermediate portions disposed between the first end portions and the second end portions, wherein the first end portions and the second end portions are coupled to the struts, wherein the intermediate portions are radially aligned with the first end portions and the second end portions when the valve frame is in the radially compressed configuration, and wherein the intermediate portions are radially offset relative to the first end portions and the second end portions when the valve frame is in the radially expanded configuration.

Example 79. The prosthetic heart valve of any example herein, particularly example 77 or example 78, wherein the intermediate portions of the anchoring members are configured to buckle when the valve frame moves from the radially compressed configuration to the radially expanded configuration.

Example 80. The prosthetic heart valve of any example herein, particularly example 78 or example 79, wherein the anchoring members include one or more first anchoring members and one or more second anchoring members, wherein the first anchoring members extend radially outwardly when the valve frame is in the radially expanded configuration, and wherein the second anchoring members extend radially inwardly when the valve frame is in the radially expanded configuration.

Example 81. The prosthetic heart valve of any example herein, particularly example 80, wherein the first anchoring members are disposed at an inflow end portion of the valve frame, and wherein the second anchoring members are disposed at an outflow end portion of the valve frame.

Example 82. The prosthetic heart valve of any example herein, particularly example 80, wherein the first anchoring members are disposed at an outflow end portion of the valve frame, and wherein the second anchoring members are disposed at an inflow end portion of the valve frame.

Example 83. The prosthetic heart valve of any example herein, particularly example 80, wherein the first anchoring members are disposed at an inflow end portion of the valve frame, and wherein the second anchoring members are disposed at an inflow end portion of the valve frame.

Example 84. The prosthetic heart valve of any example herein, particularly example 80, wherein the first anchoring members are disposed at an outflow end portion of the valve frame, and wherein the second anchoring members are disposed at an outflow end portion of the valve frame.

Example 85. A prosthetic heart valve comprises an inner frame, a valvular structure, and an outer frame. The inner frame comprises a plurality of struts. The inner frame is expandable from a radially compressed delivery configuration to a radially expanded configuration. The valvular structure comprises a plurality of leaflets. The valvular structure is disposed radially within and is coupled to the inner frame. The valvular structure is configured to allow blood flow through the prosthetic heart valve in a first direction and to restrict blood flow through the prosthetic heart valve in a second direction. The outer frame comprises a plurality of struts. The outer frame is disposed radially outwardly from and is coupled to the inner frame. The struts of the outer frame comprise one or more tissue-engaging elements configured for contacting native tissue to help secure the prosthetic heart valve at an implantation location.

Example 86. The prosthetic heart valve of any example herein, particularly example 85, wherein the inner frame comprises a first thickness, wherein the outer frame comprises a second thickness, and wherein the second thickness is less than the first thickness.

Example 87. A method of implanting the prosthetic heart valve assembly, the prosthetic heart valve assembly, the prosthetic heart valve, or the anchoring frame of any example herein, particularly any one of examples 1-86, wherein the prosthetic heart valve assembly, the prosthetic heart valve, or the anchoring frame are positioned in a native aortic valve annulus.

Example 88. A method of implanting the prosthetic heart valve assembly, the prosthetic heart valve assembly, the prosthetic heart valve, or the anchoring frame of any example herein, particularly any one of examples 1-86, wherein the prosthetic heart valve assembly, the prosthetic heart valve, or the anchoring frame are positioned in a native mitral valve annulus.

Example 89. A method of implanting a prosthetic heart valve of any example herein, particularly example 87 or example 88, the method further comprising releasably coupling the prosthetic heart valve or the prosthetic heart valve assembly to a delivery apparatus, inserting the delivery apparatus and the prosthetic heart valve or the prosthetic heart valve assembly into a patient's vasculature, advancing the prosthetic heart valve or the prosthetic heart valve assembly to an implantation location, expanding the prosthetic heart valve or the prosthetic heart valve assembly from the radially compressed configuration to the radially expanded configuration, wherein the prosthetic heart valve or the prosthetic heart valve assembly contacts native tissue at the implantation location, and releasing the prosthetic heart valve or the prosthetic heart valve assembly from the delivery apparatus.

Example 90. A prosthetic heart valve comprises an inner frame, a valvular structure, and an outer frame. The inner frame comprising a plurality of struts. The inner frame is expandable from a radially compressed delivery configuration to a radially expanded configuration, wherein the inner frame comprises a first thickness. The valvular structure comprising a plurality of leaflets. The valvular structure is disposed radially within and is coupled to the inner frame. The valvular structure is configured to allow blood flow through the prosthetic valve in a first direction and to restrict blood flow through the prosthetic valve in a second direction. The outer frame comprising a plurality of struts. The outer frame is disposed radially outwardly from and is coupled to the inner frame. The struts of the outer frame comprise one or more tissue-engaging elements configured for contacting native tissue to help secure the prosthetic valve at an implantation location. The outer frame comprises a second thickness which is less than the first thickness of the inner frame.

Example 91. The prosthetic valve of any example herein, particularly example 90, wherein the prosthetic valve is configured to be implanted in a blood vessel.

Example 92. The prosthetic valve of any example herein, particularly example 91, wherein the prosthetic valve is configured to be implanted in a vena cava.

Example 93. A docking assembly comprises a prosthetic docking device and an anchoring frame. The prosthetic docking device including a frame configured for receiving and supporting a prosthesis therein. The anchoring frame coupled to the frame of the prosthetic docking device and comprising a plurality of interconnected struts and a plurality of projections extending from the plurality of interconnected struts. The prosthetic docking device and the anchoring frame are configured to be moved from a radially compressed configuration to a radially expanded configuration. The projections of the anchoring frame are configured to engage native tissue at an implantation location to help prevent the assembly from moving relative to the implantation location.

Example 94. The docking assembly of any example herein, particularly example 93, wherein the prosthesis is a prosthetic heart valve.

Example 95. An assembly comprises a prosthetic device, a first anchoring frame, and a second anchoring frame. The prosthetic docking device including a frame configured for receiving and supporting a prosthesis therein. The frame comprises a first end portion and a second end portion. The first anchoring frame is coupled to first end portion of the frame of the prosthetic docking device and comprises a first plurality of interconnected struts and a first plurality of projections extending from the first plurality of interconnected struts. The second anchoring frame is coupled to the frame of the prosthetic docking device and comprises a second plurality of interconnected struts and a second plurality of projections extending from the second plurality of interconnected struts.

Example 96. The assembly of any example herein, particularly example 95, wherein the first anchoring frame is spaced axially from the second anchoring frame.

Example 97. An assembly comprises a prosthetic heart valve, a first anchoring frame, and a second frame. The prosthetic heart valve including a frame and a valvular structure supported within the frame. The frame comprises a first end portion and a second end portion. The first anchoring frame coupled to first end portion of the frame of the prosthetic heart valve and comprising a first plurality of interconnected struts and a first plurality of projections extending from the first plurality of interconnected struts. The second anchoring frame coupled to the frame of the prosthetic heart valve and comprising a second plurality of interconnected struts and a second plurality of projections extending from the second plurality of interconnected struts.

Example 98. The assembly of any example herein, particularly example 97, wherein the first anchoring frame is spaced axially from the second anchoring frame.

Example 99. An assembly comprises a graft, a first anchoring frame, and a second anchoring frame. The graft including a frame that is radially expandable within a blood vessel. The first anchoring frame coupled to first end portion of the frame of the graft and comprising a first plurality of interconnected struts and a first plurality of projections extending from the first plurality of interconnected struts. The second anchoring frame coupled to the frame of the graft and comprising a second plurality of interconnected struts and a second plurality of projections extending from the second plurality of interconnected struts.

Example 100. The assembly of any example herein, particularly example 99, wherein the first anchoring frame is spaced axially from the second anchoring frame.

Example 101. An assembly comprises a graft and an anchoring frame. The graft including a frame that is radially expandable within a blood vessel. The anchoring frame coupled to the frame of the graft and comprising a plurality of interconnected struts and a plurality of projections extending from the plurality of interconnected struts. The graft and the anchoring frame are configured to be moved from a radially compressed configuration to a radially expanded configuration. The projections of the anchoring frame are configured to engage native tissue at an implantation location to help prevent the assembly from moving relative to the implantation location.

Example 102. A prosthetic heart valve assembly comprises a prosthetic heart valve, an anchoring frame, and one or more sutures. The prosthetic heart valve comprising a valve frame and a valvular structure. The valve frame comprises a plurality of struts. The valve frame is expandable from a radially compressed delivery configuration to a radially expanded configuration. The valvular structure comprising a plurality of leaflets. The valvular structure is disposed radially within and is coupled to the valve frame. The valvular structure is configured to allow blood flow through the prosthetic heart valve assembly in a first direction and to restrict blood flow through the prosthetic heart valve assembly in a second direction. The anchoring frame comprising a plurality of struts. The anchoring frame is disposed radially outwardly from and is coupled to the valve frame. The struts of the anchoring frame comprise one or more tissue-engaging elements configured for contacting native tissue to help secure the prosthetic heart valve assembly at an implantation location. The one or more sutures extend circumferentially around the anchoring frame and configured for reducing paravalvular leakage between the native tissue and the prosthetic heart valve.

Example 103. The prosthetic heart valve assembly of any example herein, particularly example 102, wherein the one or more sutures comprises a first suture and a second suture, and wherein the first suture is spaced axially from the second suture.

Example 104. The prosthetic heart valve assembly of any example herein, particularly example 102 or examples 103, wherein the one or more sutures are wrapped around the struts of the anchoring frame.

Example 105. The prosthetic heart valve assembly of any example herein, particularly any one of examples 102-104, wherein the one or more sutures are wrapped around the struts of the valve frame.

Example 106. The prosthetic heart valve assembly of any example herein, particularly any one of examples 102-105, wherein the one or more sutures comprise texturized filaments that are braided together.

Example 107. The prosthetic heart valve assembly of any example herein, particularly any one of examples 102-106, wherein the one or more sutures comprise texturized filaments that are twisted together.

Example 108. The prosthetic heart valve assembly of any example herein, particularly any one of examples 106-107, wherein the one or more sutures are texturized via pin texturizing.

Example 109. The prosthetic heart valve assembly of any example herein, particularly any one of examples 106-108, wherein the one or more sutures are texturized via gear texturizing.

Example 110. The prosthetic heart valve assembly of any example herein, particularly any one of examples 106-109, wherein the one or more sutures are texturized via air texturizing.

Example 111. The prosthetic heart valve assembly of any example herein, particularly any one of examples 102-110, further comprising an inner skirt disposed on the inside of the valve frame.

The features described herein with regard to any example can be combined with other features described in any one or more of the other examples, unless otherwise stated. For example, any one or more of the features of the anchoring frame 104 can be combined with any one or more of the features of the anchoring frame 900, and vice versa. As another example, any of the frames (e.g., the frames 100, 400, 502, 602, 800, 900, 1100, 1200, 1500, 1600, 1700, and/or 1800) can be used with one or more of the tissue-engaging elements (e.g., the tissue-engaging elements 1302 and/or 1402).

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic heart valve assembly comprising:
a prosthetic heart valve comprising:
a valve frame comprising a plurality of struts, wherein the valve frame is expandable from a radially compressed delivery configuration to a radially expanded configuration; and
a valvular structure comprising a plurality of leaflets, wherein the valvular structure is disposed radially within and is coupled to the valve frame, and wherein the valvular structure is configured to allow blood flow through the prosthetic heart valve assembly in a first direction and to restrict blood flow through the prosthetic heart valve assembly in a second direction; and
an anchoring frame comprising a plurality of struts, wherein the anchoring frame is disposed radially outwardly from and is coupled to the valve frame, and wherein the struts of the anchoring frame comprise one or more tissue-engaging elements configured for contacting native tissue to help secure the prosthetic heart valve assembly at an implantation location.

2. The prosthetic heart valve assembly of claim 1, wherein the tissue-engaging elements include one or more projections extending from the struts of the anchoring frame.

3. The prosthetic heart valve assembly of claim 2, wherein the projections extend from the struts at an angle relative to a central longitudinal axis of the prosthetic heart valve assembly.

4. The prosthetic heart valve assembly of claim 2, wherein the projections extend from the struts such that the projections are at least substantially parallel to a central longitudinal axis of the prosthetic heart valve assembly.

5. The prosthetic heart valve assembly of claim 1, wherein the tissue-engaging elements include a plurality of grooves formed in the struts of the anchoring frame.

6. The prosthetic heart valve assembly of claim 5, wherein the grooves are spaced apart relative to each other so as to form ridges between the grooves.

7. The prosthetic heart valve assembly of claim 1, wherein the tissue-engaging elements include a plurality of tines extending from the struts of the anchoring frame, and wherein the tines are arranged in pairs of tines that are axially aligned with each other.

8. The prosthetic heart valve assembly of claim 7, wherein each pair of tines includes a first tine and a second tine, wherein the first tine is disposed toward an inflow end portion of the prosthetic heart valve assembly relative to the second tine, wherein the first tine and the second tine are spaced apart by a first distance when the anchoring frame is in a radially compressed configuration, wherein the first tine and the second tine are spaced apart by a second distance when the anchoring frame is in a radially expanded configuration, and wherein the second distance is less than the first distance.

9. The prosthetic heart valve assembly of claim 8, wherein the anchoring frame comprises a plurality of cells, wherein the cells have a first axial length when the anchoring frame is in the radially compressed configuration and a second axial length when the anchoring frame is in the radially expanded configuration, the second axial length being less than the first axial length, wherein each pair of tines is disposed within a respective cell, and wherein a combined axial length of the first tine and the second tine of each pair of tines is less than the second axial length of the respective cell in which the pair of tines is disposed.

10. The prosthetic heart valve assembly of claim 1, wherein the tissue-engaging elements include a plurality of tines, wherein the tines are configured to be radially aligned with the struts of the anchoring frame when the anchoring frame is in a radially compressed configuration, and wherein the tines are configured to extend radially outwardly from the struts of the anchoring frame when the anchoring frame is in a radially expanded configuration.

11. The prosthetic heart valve assembly of claim 10, wherein the anchoring frame comprises a plurality of cells, wherein the cells have a first axial length when the anchoring frame is in the radially compressed configuration and a second axial length when the anchoring frame is in the radially expanded configuration, the second axial length being less than the first axial length, and wherein the tines have an axial length that is less than the first axial length of the cells and greater than the second axial length of the cells.

12. The prosthetic heart valve assembly of claim 11, wherein the tines axially overlap with the struts of the anchoring frame that define the cells when the anchoring frame is in the radially expanded configuration.

13. The prosthetic heart valve assembly of claim 10, wherein the struts of the anchoring frame comprise one or more ramped surfaces configured to direct the tines radially outwardly relative to the struts as the anchoring frame moves from the radially compressed configuration to the radially expanded configuration.

14. The prosthetic heart valve assembly of claim 10, wherein the tines comprise ramped surfaces configured to direct the tines radially outwardly relative to the struts as the anchoring frame moves from the radially compressed configuration to the radially expanded configuration.

15. The prosthetic heart valve assembly of claim 10, wherein each of the tines comprises a fixed end portion and a free end portion.

16. An anchoring frame for a prosthetic heart valve assembly, the anchoring frame comprising:
a plurality of interconnected struts configured to be moved from a radially compressed configuration to a radially expanded configuration, wherein the struts are configured to be coupled to a prosthetic heart valve; and
a plurality of projections extending from the struts, wherein the projections are configured to engage native tissue at an implantation location.

17. The anchoring frame of claim 16, wherein one or more of the projections comprises a spike.

18. The anchoring frame of claim 16, wherein one or more of the projections comprises a ball-shaped bulge.

19. An anchoring frame for a prosthetic heart valve assembly, the anchoring frame comprising a plurality of interconnected struts comprising a plurality of grooves formed in radially outwardly facing surfaces of the struts,
wherein the struts are configured to be coupled to a prosthetic heart valve,
wherein the anchoring frame can be expanded from a radially compressed configuration to a radially expanded configuration, and
wherein the grooves are configured to receive native tissue therein when the anchoring frame is radially expanded at an implantation location.

20. The anchoring frame of claim 19, wherein the grooves are spaced apart from each other such that the struts comprise ridges between the grooves.
